(19) United States
(12) Patent Application Publication
(10) Pub. No.: US 2014/0235500 A1
(43) Pub. Date: Aug. 21, 2014

(54) HIGH THROUGHPUT INSTRUMENTATION TO SCREEN CELLS AND PARTICLES BASED ON THEIR MECHANICAL PROPERTIES

(71) Applicant: The Regents of the University of California, Oakland, CA (US)

(72) Inventors: Amy Rowat, Santa Monica, CA (US); Dongping Qi, Los Angeles, CA (US)

(73) Assignee: The Regents of the University of California, Oakland, CA (US)

(21) Appl. No.: 14/351,115

(22) PCT Filed: Oct. 15, 2012
(86) PCT No.: PCT/US2012/060297

§ 371 Date: Apr. 10, 2014

Related U.S. Application Data
(60) Provisional application No. 61/548,124, filed on Oct. 17, 2011.
(60) Provisional application No. 61/547,703, filed on Oct. 15, 2011.

(51) Int. Cl. G01N 33/50

(52) U.S. Cl. CPC G01N 33/5091; G01N 33/5005; G01N 33/5011; G01N 2500/10
USPC 506/10; 506/39; 435/366; 435/255.1; 435/254.1; 435/348; 435/252.1; 435/257.1; 435/410

(57) ABSTRACT

A system for the high-throughput screening of mechanical properties of cells and/or particles is provided. In certain embodiments the system comprises a first structure comprising a plurality of sample receiving wells; a second structure comprising a plurality of exit ports; a porous structure disposed between said plurality of sample receiving wells and said plurality of exit ports and in communication with said plurality of sample receiving wells and in communication with said plurality of exit ports, and said first structure, said second structure and said porous structure are sealed or configured such that when sufficient pressure is applied to sample receiving wells in said first structure said cells or particles migrate through the porous structure into said exit ports, and wherein the mean or the median pore size of said porous structure is smaller than the mean or median cross-section of a cell or particle that is to be screened.

### CROSS-REFERENCE TO RELATED APPLICATIONS

This application claims benefit of and priority to U.S. Ser. No. 61/547,703, filed on Oct. 15, 2011, and to U.S. Ser. No. 61/548,124, filed on Oct. 17, 2011, both of which are incorporated herein by reference in their entirety for all purposes.

### STATEMENT OF GOVERNMENTAL SUPPORT

Not Applicable

### BACKGROUND

Cells are viscoelastic materials whose mechanical properties are implicated in a wide range of biological and disease contexts, such as cell motility, metastasis, as well as how cells sense physical forces. Cells sense and respond to the stiffness of their surroundings and undergo dramatic changes in shape. However, cells can also maintain stable shapes with structural integrity and are the building units of tissues and organs. The mechanical properties of cells and nuclei are critical to their function. Cytoskeletal proteins in the cytoplasm are major contributors to cell mechanical properties. However the nucleus typically occupies a significant volume of the cell, and is about ten-fold stiffer than the surrounding cytoplasm; therefore, the nucleus is also a major determinant of whole cell deformability in a range of physiological contexts (Rowat et al. (2008) BioEssays, 30: 226-36; Friedl et al. (2011) Curr. Opin. Cell. Biol., 23: 55-64). For example, neutrophil cells have irregular, multi-lobed, nuclei that facilitate the transit of these cells through narrow pores as required for immune function (see, e.g., Erzurum et al. (1991) Am. J. Respir. Cell Mol. Biol. 5: 230-341). The degree of nuclear shape irregularity in cancer cells is widely used for disease prognosis and diagnosis (Bloom and Richardson (1957) Br. J. Cancer, 11: 359-377). Moreover, the elastic modulus of malignant cells correlates with their invasive potential (Xu et al. (2012) PLoS ONE 7: e46609; Swaminathan et al. (2011) Cancer Res. 71:5075-80). Despite the biomedical implications of cellular and nuclear shape stability, the molecular and physical origins of these properties are poorly understood.

Our understanding of the molecular origins of cellular and nuclear mechanical properties is still very limited. Much of our knowledge results from studies of a few specific cytoskeletal or nuclear envelope proteins, such as the intermediate filament protein lamin A. Knowledge of the effects of lamin A is strongly motivated by the wide spectrum of ‘laminopathy’ diseases that can arise from point mutations in the gene encoding this single protein (see, e.g., Rowat et al. (2008) BioEssays, 30: 226-236; Dechat et al. (2008) Genes Dev. 22: 832-853). Such studies provide insight into nuclear structure and mechanics, however, the mechanical properties of biopolymer networks in cells typically depend upon the interactions and cooperative contributions of multiple proteins (Kasza et al. (2007) Curr. Opin. Cell Biol. 19: 101-107). Indeed, there are over one hundred nuclear envelope proteins identified (Schirmer et al. (2003) Science, 301: 1380-1382), yet lamin A remains a prime target of mechanical studies and the extent to which other proteins collectively regulate nuclear shape stability remains unclear. For example, there is emerging evidence that histone protein modifications can also impact whole cell deformability. To develop a mechanistic, molecular-level understanding of an observable phenotype, RNAi is a powerful way to generate thousands of distinct cell variants where each lacks the expression of a specific protein. However, a method to efficiently screen the nuclear, or even cellular, mechanical phenotype of thousands of variants does not exist.

### SUMMARY

In various embodiments a system for the high-throughput screening of mechanical properties of cells and/or particles is provided. In certain embodiments the system comprises a first plate or layer comprising a first plurality of wells; a second plate or layer comprising a second plurality of wells where the second layer is aligned with the first layer such that wells in the first layer align with wells in the second layer. A porous layer is disposed between the first layer and the second layer. Pressure is applied to the wells comprising the first layer and the rate of cells' or particles' passage into wells of the second layer provides a measure of the mechanical properties of the cells or particles.

In various embodiments a system for high-throughput screening of mechanical properties of cells and/or particles is provided. IN some embodiments, the system comprises a first structure comprising a plurality of sample receiving wells; a second structure comprising a plurality of exit ports; a porous structure disposed between the plurality of sample receiving wells and the plurality of exit ports and in communication with the plurality of sample receiving wells and in communication with the plurality of exit ports, and the first structure, the second structure and the porous structure are sealed or configured such that when sufficient pressure is applied to sample receiving wells in the first structure the cells or particles migrate through the porous structure into the exit ports, and where the mean or the median pore size of the porous structure is smaller than the mean or median cross-section of a cell or particle that is to be screened. In some embodiments the porous structure is fabricated as a component of the first structure. In some embodiments the porous structure fabricated is as a component of the second structure. In some embodiments the porous structure is provided as a third structure sandwiched between the first structure and the second structure. In some embodiments the first structure, the porous structure, and second structure are fabricated as a single unitary structure. In some embodiments the system further comprises a loading plate comprising a plurality of loading wells where the loading plate is disposed on the first structure to align loading wells with the sample receiving wells and permit transfer of one or more samples from the wells comprising the loading plate to wells comprising the plurality of sample receiving wells. In some embodiments the system further comprises one or a plurality of micropipette tips where the tips are each inserted into one of the wells comprising the plurality of sample receiving wells. In some embodiments the system is configured so there is substantially no gas or fluid leakage between the first structure, the porous structure, and the second structure other than from the receiving wells, through the porous structure, into the exit ports. In some embodiments the first structure comprise at least 48 receiving wells, or at least 96 receiving wells, or at least 384 receiving wells. In some embodiments the first structure is fabricated from a material selected from the group consisting of a plastic, a soft lithography material, a ceramic, and glass. In some embodiments the second structure is fabricated from a material selected from the group consisting of a plastic, an elastomeric material, a ceramic, and glass. In some embodiments the porous structure is fabricated from a material selected from the group consisting of a plastic, an elastomeric material, a ceramic, and glass. In some embodiments the porous structure comprises a porous membrane. In some embodiments the porous structure is fabricated from a soft lithography material (e.g., polydimethylsiloxane (PDMS), polyolefin plastomers (POPs), perfluoropolyethylene (a-PFPE), polyurethane, polyimides, and cross-linked NOVOLAC® (phenol formaldehyde polymer) resin, and the like). In some embodiments the soft lithography material comprises PDMS. In some embodiments the porous structure comprises an array of posts. In some embodiments the porous structure comprises a collection of channels. In some embodiments the porous structure comprises a bed of particles. In some embodiments the first structure is fabricated from a soft lithography material. In some embodiments the second structure is fabricated from a soft lithography material (e.g., polydimethylsiloxane (PDMS), polyolefin plastomers (POPs), perfluoropolyethylene (a-PFPE), polyurethane, polyimides, and cross-linked NOVOLAC® (phenol formaldehyde polymer) resin, and the like). In some embodiments the soft lithography material comprises PDMS. In some embodiments the porous structure comprises a mean or median pore size that ranges from about 0.1 μm, or about 0.5 μm, or about 1 μm, or about 3 μm, or about 5 μm, up to about 500 μm, or up to about 400 μm, or up to about 300 μm, or up to about 200 μm, or up to about 150 μm, or up to about 100 μm, or up to about 50 μm, or up to about 40 μm, or up to about 30 μm, or up to about 20 μm, or up to about 15 μm, or up to about 10 μm. In some embodiments the porous structure comprises a mean or median pore size that ranges from about 1 μm to about 50 μm. In some embodiments the porous structure comprises a mean or median pore size that ranges from about 2 μm to about 30 μm. In some embodiments the porous structure comprises a mean or median pore size that ranges from about 3 μm to about 10 μm. In some embodiments the porous structure comprises a mean pore size of about 5 μm. In various embodiments mean or median pore sizes can be greater or smaller than those identified above. It will be appreciated that in various embodiments, “pore size” refers to a characteric dimension of a pore, channel, interstice and the like through which the cell or particle must transit to pass through the porous structure. Thus, for example in certain embodiments, the pore size can be given as a pore or channel diameter or width. In some embodiments the system further comprises a lid comprising a port to permit entry of a pressurized liquid or gas, where the lid is sealed to the first structure to permit pressurization wells comprising the first plurality of wells. In some embodiments the wells are pressurized by a gas. In some embodiments, the gas pressure is regulated by an electro-pneumatic pressure regulator, or some other source of pressure regulation (e.g. a manometer). In some embodiments the pressure regulator is under computer control. In certain embodiments sample receiving wells contain eukaryotic cells. In certain embodiments sample receiving wells contain mammalian cells (e.g., human cells). In certain embodiments sample receiving wells contain cells selected from the group consisting of yeast cells, fungal cells, insect cells, bacterial cells, algal cells, plant cells, and mammalian cells. In certain embodiments sample receiving wells are coated with bovine serum albumin (BSA). In certain embodiments sample receiving wells are coated with a surfactant (e.g., an anionic surfactant, a cationic surfactant, an amphoteric surfactant, a non-ionic surfactant, etc.). In some embodiments the surfactant is Pluronic F127. In certain embodiments different sample receiving wells contain different test agents or different combinations of test agents, or cells exposed to different test agents or combinations of different test agents. In some embodiments the test agents comprise shRNA, or a small organic molecule.

In certain embodiments methods for detecting differences in the mechanical properties of cells or particles are provided where the methods comprise providing a system for HTMS screening as described and/or claimed herein; placing the cells or particles in wells comprising the plurality of sample receiving wells; pressurizing the wells containing the cells or particles; collecting and/or detecting cells or particles that pass through the porous structure into (or through) the receiving ports and/or collecting and/or detecting cells or particles that are retained in the first plurality of wells; and quantifying the number of cells or particles that pass through the porous structure into the exit ports and/or that are retained in the receiving wells where the number of cells or particles retained in the receiving wells and/or that pass through into the exit ports provides a measure of the stiffness of the cells or particles. In some embodiments the quantifying comprises quantifying the number of cells or particles that pass through the porous structure and into or through the exit port(s). In some embodiments the quantifying comprises quantifying the number of cells or particles that are retained in the sample receiving wells. In some embodiments the pressurizing comprises placing a lid comprising a port to permit entry of a pressurized liquid or gas over wells comprising the sample receiving wells, where the lid is sealed to the first structure to permit pressurization of the sample receiving wells. In some embodiments the wells are pressurized by a gas. In some embodiments the gas pressure is regulated by an electro-pneumatic pressure regulator. In some embodiments the pressure regulator is under computer control. In some embodiments, the cells or particles comprise cells (e.g., eukaryotic cells or prokaryotic cells). In some embodiments, the cells or particles comprise mammalian cells (e.g., human cells). In some embodiments, the cells or particles comprise pathological cells (e.g., cancer cells). In some embodiments, the cells or particles comprise cells selected from the group consisting of yeast cells, fungal cells, insect cells, bacterial cells, algal cells, plant cells, and mammalian cells. In various embodiments sample receiving wells are coated with bovine serum albumin (BSA). In various embodiments sample receiving wells are coated with a surfactant (e.g., non-ionic surfactant, a cationic surfactant, an anionic surfactant, and an amphoteric surfactant). In some embodiments the surfactant is Pluronic F127. In some embodiments, different sample receiving wells contain different test agents or different combinations of test agents. In some embodiments the test agents comprise shRNA, or a small organic molecule. In some embodiments the test agents comprise pharmaceuticals. In some embodiments the test agents comprise anti-cancer pharmaceuticals or compounds believed to have anti-cancer activity. Illustrative anti-cancer compounds include, but are not limited to anti-cancer antibodies, small molecules targeting IGF1R, small molecules targeting EGFR receptor, small molecules targeting ErbB2, small molecules targeting cMET, antimetabolites, alkylating agents, topoisomerase inhibitors, microtubule targeting agents, kinase inhibitors, protein synthesis inhibitors, somatostatin analogs, glucocorticoids, aromatose inhibitors, mTOR inhibitors, protein Kinase B (PKB) inhibitors, phosphatidylinositol, 3-Kinase (PI3K) Inhibitors, cyclin dependent kinase inhibitors, anti-TRAIL molecules, MEK inhibitors, and the like. In certain embodiments the anti-cancer compounds include, but are not limited to flourouracil (5-FU), capecitabine/XELODA, 5-Trifluoromethyl-2′-deoxyuridine, methotrexate sodium, raltitrexed/Tomudex, pemetrexed/Alimta®, cytosine Arabinoside (Cytarabine, Ara-C)/Thioguanine, 6-mercaptopurine (Mercaptopurine, 6-MP), azathioprine/Azasan, 6-thioguanine (6-TG)/Purinethol (TEVA), pentostatin/Nipent, fludarabine phosphate/Fludara®, cladribine (2-CdA, 2-chlorodeoxyadenosine)/Leustatin, floxuridine (5-fluoro-2)/FUDR (Hospira, Inc.), ribonucleotide Reductase Inhibitor (RNR), cyclophosphamide/Cytoxan (BMS), neosar, ifosfamide/Mitoxana, thiotepa, BCNU -- 1,3-bis(2-chloroethyl)-1-nitosourea, 1,-(2-chloroethyl)-3-cyclohexyl-Initrosourea, methyl CCNU, hexamethylmelamine, busulfan/Myleran, procarbazine HCL/Matulane, dacarbazine (DTIC), chlorambucil/Leukaran®, melphalan/Alkeran, cisplatin (Cisplatinum, CDDP)/Platinol, carboplatin/Paraplatin, oxaliplatin/Eloxitan, bendamustine, carmustine, chloromethine, dacarbazine (DTIC), fotemustine, lomustine, mannosulfan, nedaplatin, nimustine, prednimustine, ranimustine, satraplatin, semustine, streptozocin, temozolomide, treosulfan, triaziquone, triethylene melamine, thioTEPA, triplatin tetranitrate, trofosfamide, uramustine, doxorubicin HCL/Doxil, daunorubicin citrate/Daunoxome®, mitoxantrone HCL/Novantrone, actinomycin D, etoposide/Vepesid, topotecan HCL/Hycamtin, teniposide (VM-26), irinotecan HCL(CPT-11)/, camptosar®, camptothecin, Belotecan, rubitecan, vincristine, vinblastine sulfate, vinorelbine tartrate, vindesine sulphate, paclitaxel/Taxol, docetaxel/Taxotere, nanoparticle paclitaxel, abraxane, ixabepilone, larotaxel, ortataxel, tesetaxel, vinflunine, and the like. In certain embodiments the anti-cancer drug(s) comprise one or more drugs selected from the group consisting of carboplatin(e.g., PARAPLATIN®), Cisplatin (e.g., PLATINOL®, PLATINOL-AQ®), Cyclophosphamide (e.g., CYTOXAN®, NEOSAR®), Docetaxel (e.g., TAXOTERE®), Doxorubicin (e.g., ADRIAMYCIN®), Erlotinib (e.g., TARCEVA®), Etoposide (e.g., VEPESID®), Fluorouracil (e.g., 5-FU®), Gemcitabine (e.g., GEMZAR®), imatinib mesylate (e.g., GLEEVEC®), Irinotecan (e.g., CAMPTOSAR®), Methotrexate (e.g., FOLEX®, MEXATE®, AMETHOPTERIN®), Paclitaxel (e.g., TAXOL®, ABRAXANE®), Sorafinib (e.g., NEXAVAR®), Sunitinib (e.g., SUTENT®), Topotecan (e.g., HYCAMTIN®), Vinblastine (e.g., VELBAN®), Vincristine (e.g., ONCOVIN®, VINCASAR PFS®). In certain embodiments the anti-cancer drug comprises one or more drugs selected from the group consisting of retinoic acid, a retinoic acid derivative, doxirubicin, vinblastine, vincristine, cyclophosphamide, ifosfamide, cisplatin, 5-fluorouracil, a camptothecin derivative, interferon, tamoxifen, and taxol. In some embodiments the quantifying comprises utilizing a flow cytometer to quantify the number of cells in wells comprising the first plurality of wells and/or in wells comprising the second plurality wells. In some embodiments the flow cytometer detects a fluorescent marker in the cells. In some embodiments the quantifying comprises utilizing a microplate reader to quantify the number of cells in sample receiving wells or in wells formed from or fed by the exit port(s). In some embodiments the quantifying comprises utilizing an image analysis system to quantify the number of cells in sample receiving wells or in wells collecting cells from said exit ports.

In certain embodiments methods for determining that cells are responsive or non-responsive to a drug are provided where the methods comprise providing a system for

HTMS screening as described and/or claimed herein; placing cells to be tested in wells comprising the plurality of sample receiving wells; contacting the cells with the drug (e.g., before or after placement in the wells); pressurizing the sample receiving wells containing the cells; collecting and/or detecting cells that pass through the porous structure into the exit ports, and/or collecting and/or detecting cells that are retained in the sample receiving wells;

and quantifying the number of cells that pass through the porous structure into the exit ports and/or quantifying the number of cells that are retained in the sample receiving wells where a difference in the number of cells contacted with the drug retained in the sample receiving wells and/or that pass through into the exit ports as compared to the quantity measured for negative control cells indicates that the cells are responsive to the drug, while a substantial lack of the difference is an indicator that the drug does not alter the mechanical properties of the cells. In some embodiments the cells comprise eukaryotic cells (e.g., mammalian cells). In some embodiments the cells comprise human cells (e.g., healthy cells or pathological cells). In some embodiments the cells comprise stem cells (e.g., adult stem cells, cord blood stem cells, embryonic stem cells, induced pluripotent cells (IPSCs), etc.). In some embodiments the cells comprise cancer cells. In some embodiments the cells are derived from a tumor in a subject under treatment for a cancer. In some embodiments the cancer comprises a cancer selected from the group consisting of acute lymphoblastic leukemia (ALL), acute myeloid leukemia (AML), Adrenocortical carcinoma, AIDS-related cancers (e.g., kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, brain and spinal cord tumors, brain stem glioma, central nervous system atypical teratoid/rhabdoid tumor, central nervous system embryonal tumors, central nervous system germ cell tumors, craniopharyngioma, ependymoma, breast cancer, bronchial tumors, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm's tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer , pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor. In some embodiments the contacting comprises placing different drugs or different combinations of drugs in different wells comprising the plurality of sample receiving wells. In some embodiments the drugs comprise anti-cancer pharmaceuticals or compounds believed to have anti-cancer activity. In some embodiments, the anti-cancer drugs include, but not limited to one or more compounds selected from the group consisting of anti-cancer antibodies, small molecules targeting IGF1R, small molecules targeting EGFR receptor, small molecules targeting ErbB2, small molecules targeting cMET, antimetabolites, alkylating agents, topoisomerase inhibitors, microtubule targeting agents, kinse inhibitors, protein sysnthesis inhibitors, somatostatin analogs, glucocorticoids, aromatose inhibitors, mTOR inhibitors, protein Kinase B (PKB) inhibitors, phosphatidylinositol, 3-Kinase (PI3K) Inhibitors, cyclin dependent kinase inhibitors, anti-TRAIL molecules, MEK inhibitors, and the like. In certain embodiments the anti-cancer drug(s) comprise one or more drugs selected from the group consisting of flourouracil (5-FU), capecitabine/XELODA, 5-Trifluoromethyl-2′-deoxyuridine, methotrexate sodium, raltitrexed/Tomudex, pemetrexed/Alimta®, cytosine Arabinoside (Cytarabine, Ara-C)/Thioguanine, 6-mercaptopurine (Mercaptopurine, 6-MP), azathioprine/Azasan, 6-thioguanine (6-TG)/Purinethol (TEVA), pentostatin/Nipent, fludarabine phosphate/Fludara®, cladribine (2-CdA, 2-chlorodeoxyadenosine)/Leustatin, floxuridine (5-fluoro-2)/FUDR (Hospira, Inc.), ribonucleotide Reductase Inhibitor (RNR), cyclophosphamide/Cytoxan (BMS), neosar, ifosfamide/Mitoxana, thiotepa, BCNU -- 1,3-bis(2-chloroethyl)-1-nitosourea, 1,-(2-chloroethyl)-3-cyclohexyl-lnitrosourea, methyl CCNU, hexamethylmelamine, busulfan/Myleran, procarbazine HCL/Matulane, dacarbazine (DTIC), chlorambucil/Leukaran®, melphalan/Alkeran, cisplatin (Cisplatinum, CDDP)/Platinol, carboplatin/Paraplatin, oxaliplatin/Eloxitan, bendamustine, carmustine, chloromethine, dacarbazine (DTIC), fotemustine, lomustine, mannosulfan, nedaplatin, nimustine, prednimustine, ranimustine, satraplatin, semustine, streptozocin, temozolomide, treosulfan, triaziquone, triethylene melamine, thioTEPA, triplatin tetranitrate, trofosfamide, uramustine, doxorubicin HCL/Doxil, daunorubicin citrate/Daunoxome®, mitoxantrone HCL/Novantrone, actinomycin D, etoposide/Vepesid, topotecan HCL/Hycamtin, teniposide (VM-26), irinotecan HCL(CPT-11)/, camptosar®, camptothecin, Belotecan, rubitecan, vincristine, vinblastine sulfate, vinorelbine tartrate, vindesine sulphate, paclitaxel/Taxol, docetaxel/Taxotere, nanoparticle paclitaxel, abraxane, ixabepilone, larotaxel, ortataxel, tesetaxel, vinflunine, and the like. In certain embodiments the anti-cancer drug(s) comprise one or more drugs selected from the group consisting of carboplatin(e.g., PARAPLATIN®), Cisplatin (e.g., PLATINOL®, PLATINOL-AQ®), Cyclophosphamide (e.g., CYTOXAN®, NEOSAR®), Docetaxel (e.g., TAXOTERE®), Doxorubicin (e.g., ADRIAMYCIN®), Erlotinib (e.g., TARCEVA®), Etoposide (e.g., VEPESID®), Fluorouracil (e.g., 5-FU®), Gemcitabine (e.g., GEMZAR®), imatinib mesylate (e.g., GLEEVEC®), Irinotecan (e.g., CAMPTOSAR®), Methotrexate (e.g., FOLEX®, MEXATE®, AMETHOPTERIN®), Paclitaxel (e.g., TAXOL®, ABRAXANE®), Sorafinib (e.g., NEXAVAR®), Sunitinib (e.g., SUTENT®), Topotecan (e.g., HYCAMTIN®), Vinblastine (e.g., VELBAN®), Vincristine (e.g., ONCOVIN®, VINCASAR PFS®). In certain embodiments the anti-cancer drug comprises one or more drugs selected from the group consisting of retinoic acid, a retinoic acid derivative, doxirubicin, vinblastine, vincristine, cyclophosphamide, ifosfamide, cisplatin, 5-fluorouracil, a camptothecin derivative, interferon, tamoxifen, and taxol.

In various embodiments methods for identifying abnormal cells in a subject, are provided where the methods comprise providing a system for HTMS screening as described and/or claimed herein; placing cells from the subject in wells comprising the plurality of sample receiving wells; pressurizing the wells containing the cells; collecting and/or detecting cells that pass through the porous structure into (or through) the exit ports, and/or collecting and/or detecting cells that are retained in sample receiving wells; and quantifying the number of cells that pass through the porous structure into (or through) the exit ports and/or quantifying the number of cells that are retained in wells comprising plurality of sample receiving wells where a difference in the number of cells that pass through or that are retained as compared to the same measurement performed on normal healthy cells is an indicator that cells from the subject are abnormal. In some embodiments the cells are further screened for one or more cancer markers. In some embodiments the cells comprise cells from a non-human mammal. In some embodiments the cells comprise cells from a human. In some embodiments the cells comprise cells from a tissue biopsy. In some embodiments the cells comprise cells from a cell culture. In some embodiments the cells are from a mammal diagnosed as having or suspected of having cancer. In some embodiments the cells comprise cancer cells.

In various embodiments of these methods quantifying comprises quantifying the number of cells that pass through the porous structure into the exit ports. In various embodiments the quantifying comprises quantifying the number of cells that are retained in wells comprising the plurality of sample receiving wells. In various embodiments the pressurizing comprises placing a lid comprising a port to permit entry of a pressurized liquid or gas over cells comprising the plurality sample receiving wells, where the lid is sealed to the first layer to permit pressurization wells comprising the plurality of sample receiving wells. In various embodiments the wells are pressurized by a gas. In various embodiments the gas pressure is regulated by an electro-pneumatic pressure regulator. In some embodiments the pressure regulator is under computer control. In various embodiments the sample receiving wells are coated with bovine serum albumin (BSA) and/or a surfactant (e.g., a cationic surfactant, an anionic surfactant, a non-ionic surfactant, an amphoteric surfactant, etc.). In some embodiments the quantifying comprises utilizing a flow cytometer to quantify the number of cells in wells comprising the plurality of sample receiving wells and/or in that pass out the exit ports. In some embodiments the flow cytometer detects a fluorescent marker in the cells. In some embodiments the quantifying comprises utilizing a microplate reader to quantify the number of cells in wells comprising the plurality of sample receiving wells and/or that pass through the exit ports. In some embodiments the quantifying comprises utilizing an image analysis system to quantify the number of cells in wells comprising the plurality of sample receiving wells and/or that are in or pass through the exit ports.

### BRIEF DESCRIPTION OF THE DRAWINGS

FIGS. 1A-1C, offer a proof of principle that cell nucleus protein levels alter the deformability of cells. FIG. 1A: Cells transiting through the channel of a microfluidic device. Scale: 10 μm. FIG. 1B: Cells expressing increased levels of lamin A take longer to deform through 5 μm-diameter constrictions, as compared to the mock-modified control. Box and whisker plot: white bars show the median deformation time, boxes show the 25th/75th-percentiles, and lines depict the 10th/90th-percentiles. FIG. 1C: Schematic illustration of our model depicting how lamin A protein expression levels impair the deformation of cells through a narrow pore in response to an applied pressure.

FIG. 2 illustrates one embodiment of the overall architecture of an HTMS system. Cells deform through the pores of a filter insert when subject to an applied pressure.

FIG. 3 illustrates the workflow of one embodiment of an illustrative HTMS experiment. The detection method illustrated here is a microplate reader, however, other detection methods including, but not limited to, plate readers, flow cytometers, and the like could be used. Lower panel shows preliminary results of drug-treated cells. Panel in step 2 shows an array of 96 pipette tips inserted into a PDMS slab (a first structure comprising a plurality of sample receiving wells).

FIG. 4 schematically illustrates one embodiment of a HTMS device 100 comprising a first structure 102 comprising a plurality of sample receiving wells 104, a second structure 108 comprising a plurality of exit ports/channels or chambers 110, and a porous structure 106 in communication (e.g., in fluid communication) with the plurality of sample receiving wells and in communication (e.g., in fluid communication) with the plurality of exit ports, channels, or chambers 110, where the porous structure 106 is disposed between the plurality of sample receiving wells 104 and said plurality of exit ports 110. The path of cells or particles through the device is shown by the block arrows. As illustrated, the sample receiving wells 104 can be offset from the positions of the exit ports 110. Porosity in the porous structure 106 is created by a series of channels 122.

FIG. 5 schematically illustrates one embodiment of an HTMS device where porosity in the porous structure 106 is created by a series of posts (pillars) 112.

FIG. 6 illustrates one illustrates one embodiment of an HTMS device where the sample receiving wells 104 are co-aligned with the exit ports 106.

FIGS. 7A and 7B illustrate embodiments of the HTMS device where the device is provided with a receptacle 118 (e.g. a microtiter plate) comprising a plurality of collection wells 126 for collecting cells or particles that pass through the porous structure. FIG. 7A illustrates the use of micropipettes 116 to deliver the cells or particles 114 into the sample receiving chambers 104, while FIG. 7B illustrates the use of a sample delivery tray or an array of wells (e.g., a loading plate) 120 comprising a plurality of sample delivery wells 128 to deliver the cells or particles 114 into the sample receiving chambers 104.

FIGS. 8A and 8B illustrates one operating configuration of an HTMS device (FIG. 8A side view, FIG. 8B top view).

FIG. 9, panels A-C, illustrates components of one embodiment of the device described herein. Panel A: A porous membrane is sandwiched between two custom machined 96-well plates. Panel B: Top-down view showing the 96-wells loaded with cell suspension. A soft rubber seal and O-rings help to achieve a pressure-tight seal. Panel C: After loading, the lid is placed on top; a pressure gauge monitors the applied pressure in the device. Scale, 5 cm.

FIGS. 10A and 10B show images of one illustrative prototype device. The figures illustrate the collection plate collection plate (panel 1 in FIGS. 10A and 10B) and its assembly into a complete device. Only a subsection of the plate is being used here: O-rings help to ensure a pressure-tight seal between the bottom collection plate and top plate. Panel 2 in FIGS. 10A and 10B illustrates the assembled device. A porous membrane is sandwiched between the bottom collection plate and top plate. Screws on the four corners of the sandwiched plates help to create a pressure-tight seal. O-rings and other sealants (including but not limited to parafilm, silicon sealants), help to create a pressure tight seal. Panel 3 in FIGS. 10A and 10B illustrate the device with the pressure chamber placed on top. A push-to-connect fitting enables easy connection to tubing that connects to a tank of compressed air outfitted with a pressure gauge. The device can be adapted to interface with a standard multiwell (e.g., 6, 12, 24, 48, 96, 384, 1536-well) plate.

FIG. 11 illustrates components of the device and a porous layer.

FIG. 12 illustrates device loading (top panel). As shown in the middle and lower panels, in certain embodiments, a rubber rim and O-rings can be used to maintain a pressure seal.

FIG. 13 illustrates one fabrication approach and various considerations.

FIG. 14 illustrates a mask used for fabricating an array of 96 porous structures.

FIG. 15 illustrates the fabrication of “posts” for a porous structure. Arrays of posts are fabricated using soft lithography on a 6″ silicon wafer. This provides a master or mold for the polydimethysiloxane (PDMS).

FIG. 16 illustrates the application of pressure to the HTMS device.

FIG. 17 shows data illustrating the effect of time on the number of cells that pass through the HTMS device when a fixed pressure is applied to the system. Representative data are shown for differentiated HL60 cells using a 5 μm isopore membrane as the “porous layer” at an applied pressure of 3.6 psi.

FIG. 18 shows data illustrating the effect of pressure on the number of cells that pass through the HTMS device. Data are provided showing the number of cells that passage through the porous layer as a function of time at two different applied pressures. Representative data are shown for differentiated HL60 cells using a 5 μm isopore membrane as the “porous layer” at applied pressures of 7.5 psi and 3.5 psi.

FIG. 19 illustrates the detection of differences between HL60 cells that are differentiated, neutrophil-type cells versus non-differentiated HL60 cells. Cells have similar size distributions, but altered mechanical properties. Scale 5 μm.

FIG. 20 illustrates the detection of differences between cells that overexpress lamin A (lamin-A modified) and cells that do not (non-modified). The number of passaged cells depends on the physical properties of the cell nucleus. Expression levels of the nuclear protein, lamin A, affect cell passage. A larger number of mock-modified cells passage through the 5 μm membrane pores in a given time compared to the lamin A-modified cells. Shown here are mean values averaged over 3 independent experiments; error bars represent SE. Scale, 5 μm.

FIG. 21 illustrates the porous membrane after cell filtration. Top panel shows cells trapped in the porous membrane. Only a fraction of the cells loaded in the HTMS device passage entirely through the porous membrane. In certain embodiments, the pores are uniformly distributed.

FIG. 22 schematically illustrates an RNAi mechanical screening experiment. Cells are treated with shRNAs that knock out particular genes. After HTMS, cells are counted using a flow cytometer, revealing hits indicate that the shRNA has altered mechanical properties of the cells. Identification of the particular shRNA indicates the gene(s) that result in the change in mechanical properties.

FIG. 23 illustrates the effect of membrane pore size on cell passage through the HTMS device using HL60 cells. The average size of HL60 cells is about 13.7 μm. When the average radius of the cell/average radius of the pore is <˜2.5 no apparent filtration was observed at this applied pressure of 0.1 psi.

FIG. 24 illustrates determination of the optimal density for an HTMS measurement. When ρ>˜1.5×10⁶ cells/ml there was pronounced pore clogging. The vertical line shows the cell density used in the experiments whose data is shown in FIGS. 25-32.

FIG. 25 shows that different cell types have distinct deformability: HL60 versus neutrophil-type (ATRA-treated) cells. The pressure dependence of cell passage is sensitive to cell stiffness. When p>p_{threshold}, filtration was observed.

FIG. 26 shows that it is possible to distinguish cell deformability at a fixed pressure. In the experiment whose results are illustrated, a 5 μm pore membrane was used and 0.3 psi was applied for 20 seconds.

FIG. 27 illustrates the effect of various anti-cancer drugs on HL60 cell mechanical properties (e.g., deformability). Without being bound by a particular theory, the drugs are believed to modify actin filaments and thereby alter the cells' mechanical properties.

FIG. 28 illustrates that HTMS measurements can be used to detect a dose response of a cancer drug treatment.

FIG. 29 illustrates the effect of various drugs on adherent murine mammary carcinoma cells (67NR).

FIG. 30 illustrates the use of HTMS measurements to detect the effect of miRNA on a cell as compared to the scrambled control. Cell size: ·17 um; 8 um pore membrane used; 0.1 psi for 40 s.

FIG. 31 illustrates the use of HTMS measurements to distinguish drug-resistant cancer cells. 10 μm pore membrane used; 0.2 psi pressure applied for 50 s.

FIG. 32 illustrates the use of HTMS measurements to detect the effects of drug treatment on cancer cells. Data: 0.4 psi for 35 s.

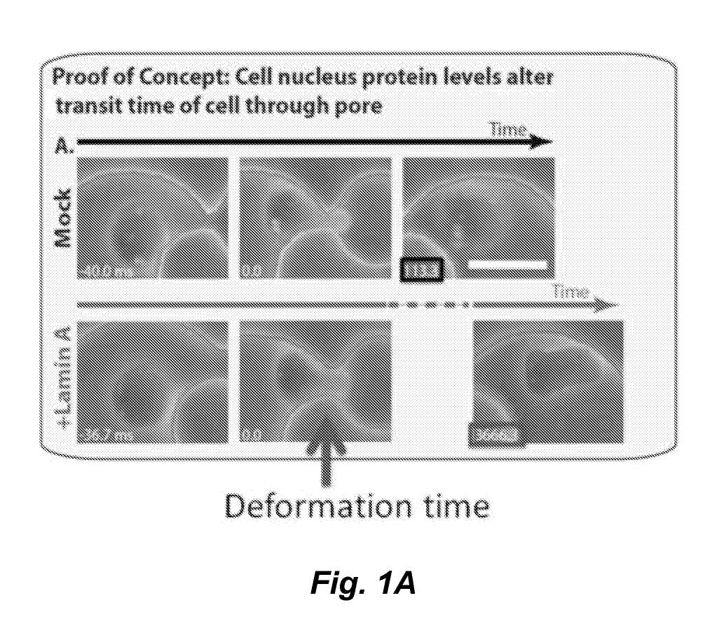

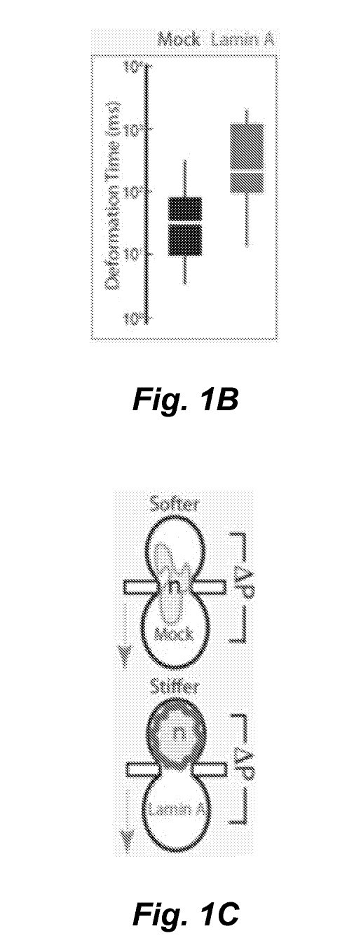

### DETAILED DESCRIPTION

In various embodiments a novel high throughput mechanical screening system (HTMS) for the effecting detection and/or quantitation of the mechanical properties of cells or particles and uses of such a system are provided. In various embodiments the HTMS systems described herein enables the measurement of cell or particle mechanical properties on 2 or more, 4 or more, 6 or more, 12 or more, 24 or more, 48 or more, 96 or more, 384 or more, or 1536 or more samples in parallel (e.g., in a microtiter plate).

In various embodiments the devices comprise a first structure (e.g., a layer or lamina) comprising a plurality of sample receiving wells juxtaposed to a porous structure that (e.g., a porous membrane, a porous zone, layer, or lamina, etc.). In some embodiments, the device can optionally further comprise a second structure (e.g., a layer or lamina) on the other side of the porous structure where the second structure comprises a plurality of exit ports. Typically the first structure, second structure and porous structure are sealed or configured such that when sufficient pressure is applied to sample receiving wells the cells or cells or particles in the receiving wells migrate through the porous structure and then out the exit ports (when present). Typically the mean or the median pore/channel size of the porous structure is smaller than the mean or median cross-section of a cell or particle that is to be screened.

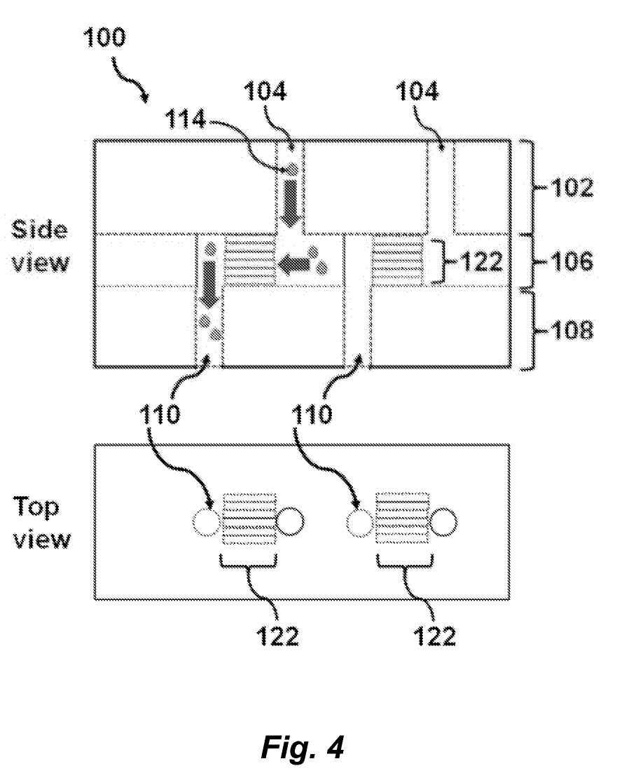

In general, the HTMS measurement methods involve disposing cells or particles on the porous structure, e.g., by placing them in receiving wells juxtaposed to the porous structure (see, e.g., FIGS. 4A 4B). The cells or particles are forced to deform through the pores due to applied external pressure. The porous structure is selected and/or fabricated so that the pore size (e.g., channel size/path dimension) requires the cells or particles to deform to pass through the porous structure. The amount of time required for the deformation depends on the mechanical properties of the interrogated sample. In certain embodiments the number of passaged cells/particles (and/or the number of cells/particles retained) is thereafter counted, and the relative numbers give a measure of relative cell or particle deformability. By selecting suitable commercially available porous membranes or by custom-fabrication of porous structures (e.g., PDMS structures) using, e.g., soft lithography, the pore depth and tortuosity of the porous structure can be tuned to enhance measurement sensitivity and dynamic range.

The HTMS measurements and systems described herein facilitate measurements and experiments that were hitherto not possible. This includes a broad range of fundamental biology studies to elucidate mechanisms of cell mechanical regulation, as well as a new class of studies using mechanical phenotype for drug and enzyme discovery with potential benefit to cancer, malaria, and biofuels research. It was discovered that different cell types can show different mechanical properties when interrogated using the HTMS systems described herein. Accordingly the devices can be used to interrogate a population of cells to identify those cells with altered properties. In some embodiments, those altered properties are indicative of a disease state.

It was also discovered that drugs, can alter the mechanical properties of cells and these alterations can be detected using the HTMS systems described herein. Accordingly the HTMS systems can be used to libraries of test agents to identify those that have an effect on the mechanical properties of cells. Conversely, cells can be screened against drugs known to alter cellular mechanical properties to identify cells that are sensitive to or resistant to the drug(s) of interest. In some embodiments, where the drugs are anti-cancer agents, the system can be used to interrogate cells to identify or characterize a particular cancer as responsive (or non-responsive) to particular drug(s) or classes of drugs.

Accordingly, in certain embodiments, methods are provided for detecting differences in the mechanical properties of cells or particles. In certain embodiments the methods comprise providing a system for the high-throughput screening of mechanical properties of cells and/or particles as described herein, placing said cells or particles in receiving wells (e.g., in wells disposed adjacent to a porous structure); pressurizing the wells containing the cells or particles; collecting and/or detecting cells or particles that pass through the porous structure into (or through) exit ports in the second structure, optionally into a second set of wells and/or collecting and/or detecting cells or particles that are retained in wells in which they were originally disposed; and quantifying the number of cells or particles that pass through the porous structure and/or that are retained in the receiving wells where the number of cells or particles retained or that pass through the porous structure provides a measure of the stiffness of the cells or particles. Typically the more easily the cells or particles are deformed and/or the greater range of deformation, the greater the number of cells or particles that pass into or through the porous structure and the fewer the number of cells or particles that are retained.

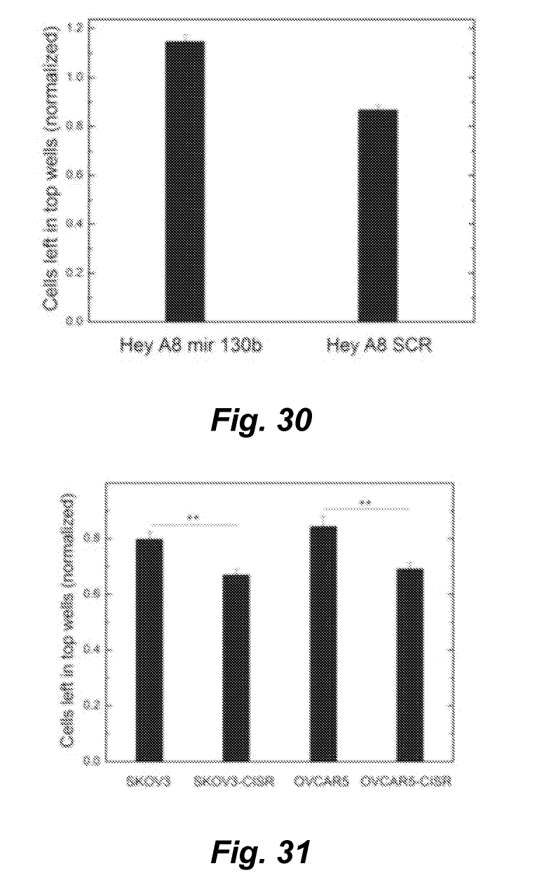

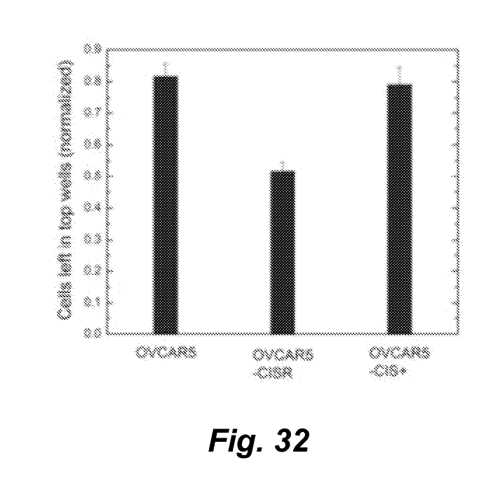

In certain embodiments, methods are provided for determining whether cells are responsive or non-responsive to a drug or therapeutic treatment (e.g., such as miRNA as illustrated in FIG. 30). In certain embodiments the methods comprise providing a system for the high-throughput screening of mechanical properties of cells and/or particles as described herein, placing said cells or particles in receiving wells (e.g., in wells disposed adjacent to a porous structure); pressurizing the wells containing the cells or particles; collecting and/or detecting cells or particles that pass through the porous structure into (or through) exit ports in the second structure, optionally into a second set of wells and/or collecting and/or detecting cells that are retained in the receiving wells in which they were originally disposed; and quantifying the number of cells or particles that pass through the porous structure and/or that are retained in the receiving wells where a difference in the number of cells contacted with the drug(s) (or administered the treatment(s)) retained or passed through the porous structure as compared to the quantity measured for negative control cells (e.g., cells not contacted with the drug(s)) or contacted with the drug(s) at a lower concentration) indicates that the cells are responsive to the drug(s), while a lack of substantial difference is an indicator that the drug does not alter the mechanical properties of the cells.

In certain embodiments, methods of identifying abnormal cells in a subject (e.g., in a human or non-human mammal) are provided. In certain embodiments the methods comprise providing a system for the high-throughput screening of mechanical properties of cells and/or particles as described herein, placing cells from the subject to be tested in receiving wells adjacent to the porous structure; pressurizing the wells containing the cells; collecting and/or detecting cells that pass through the porous structure into or through the exit ports, optionally into a second set of wells and/or collecting and/or detecting cells that are retained in the receiving wells in which they were originally disposed; and quantifying the number of cells or particles that pass through the porous structure and/or that are retained in the original receiving wells where a difference in the number of cells that pass through or that are retained as compared to the same measurement performed on normal healthy cells is an indicator that cells from the subject are abnormal.

These methods are intended to be illustrative and non-limiting. Another illustrative use of the HTMS systems and methods described herein includes, for example, RNAi mechanical screening. The ability to perform proteome-wide mechanical screen for cell and nucleus mechanical properties facilitates identification of novel candidate proteins that play a role in regulating cell and nucleus mechanical properties. In some embodiments, the HTMS systems and methods described herein can be used to probe the mechanical properties of the cell nucleus. By tuning the channel dimensions so they are small compared to the size of the nucleus, mechanical properties of the cell nucleus can be detected, showing the potential to also detect the effect of myriad molecules and point mutations that have a resultant effect on cell nucleus shape, stiffness, and/or stability. In some embodiments, the HTMS systems and methods described herein can be used for high throughput screening for drugs. Marked changes in mechanical properties of cells accompany disease. For example, red blood cells exhibit increased stiffness when infected by malaria. Exploiting these changes in mechanical properties, HTMS provides a way to screen for anti-malarial or anti-cancer compounds. In another illustrative example, mechanical properties can be used as an additional metric to quantify the effect of microRNAs on reversing cancer cell phenotype.

The methods described herein need not be limited to mammalian cells. To the contrary, use with essentially any cell is contemplated. Such cells include, but are not limited to fungal cells, insect cells, yeast cells, algal cells, bacterial cells, and the like. In one illustrative embodiment, the HTMS systems and methods described herein can be used to screen for cell-wall perturbing compounds. Compounds that perturb the cell wall of microbes such as yeast and bacteria can have antimicrobial/antibiotic properties. The systems described herein can thus be adapted to screen for new antibiotics.

The methods described herein also need not be limited to use with cells. The properties of various particles can similarly be probed. For example, in some embodiments, the HTMS systems and methods described herein can be used to probe microgel stiffness. Hydrogels are ubiquitous in the context of drug delivery and tissue engineering (Janmey et al. (2009) J. R. Soc. Interface, 6: 1-10). Their mechanical properties are critical for their biodistribution (Merkel et al. (2011) Proc. Natl. Acad. Sci. USA, 108: 586-591). HTMS can enable valuable combinatorial studies to elucidate the effects of soluble compounds on microgel stiffness.

In certain embodiments the HTMS systems and methods described herein can be used for the discovery and/or characterization of various enzymes and enzyme combinations. For example, in some embodiments, the HTMS systems and methods described herein can be used for the discovery/characterization of enzymes for use in biofuel production. Biomass-degrading enzymes can be detected by screening changes in mechanical integrity of cellulose particles. Particles can be incubated with a library of microbes secreting enzymes (or with the enzymes or enzyme cocktails). The systems described herein enables screening assays using natural substrates, that do not require additional reagents. In certain embodiments the HTMS systems and methods described herein can be used to probe liposomes and lipidic microparticles. Model lipid membranes are widely used to elucidate the mechanism of action of basic membrane molecules, such as cholesterol, as well as the action of antimicrobial peptides. Mechanical studies are commonly used to study the resultant effects on liposomes at the level of single liposomes.

Illustrative HTMS Device Configurations

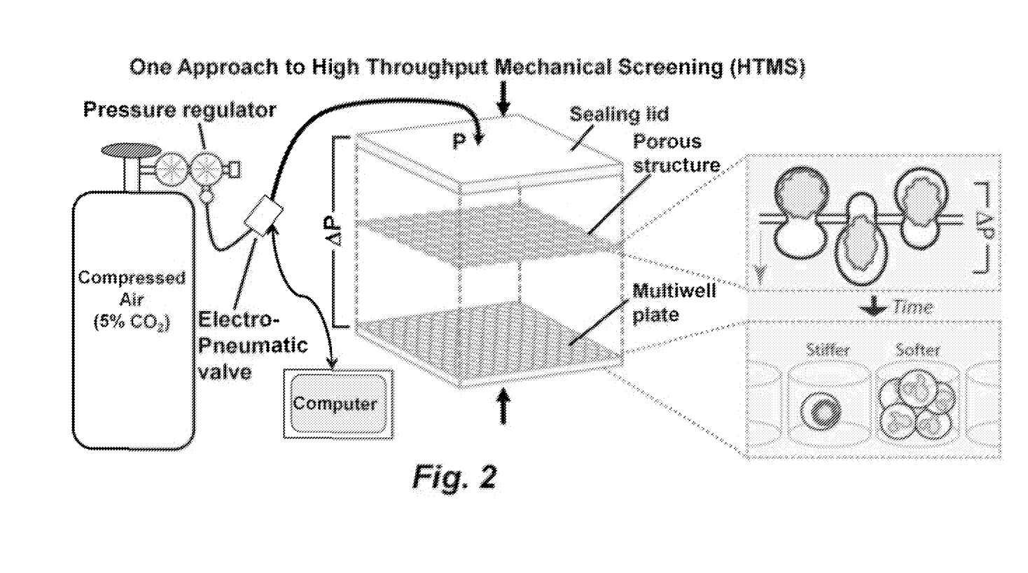

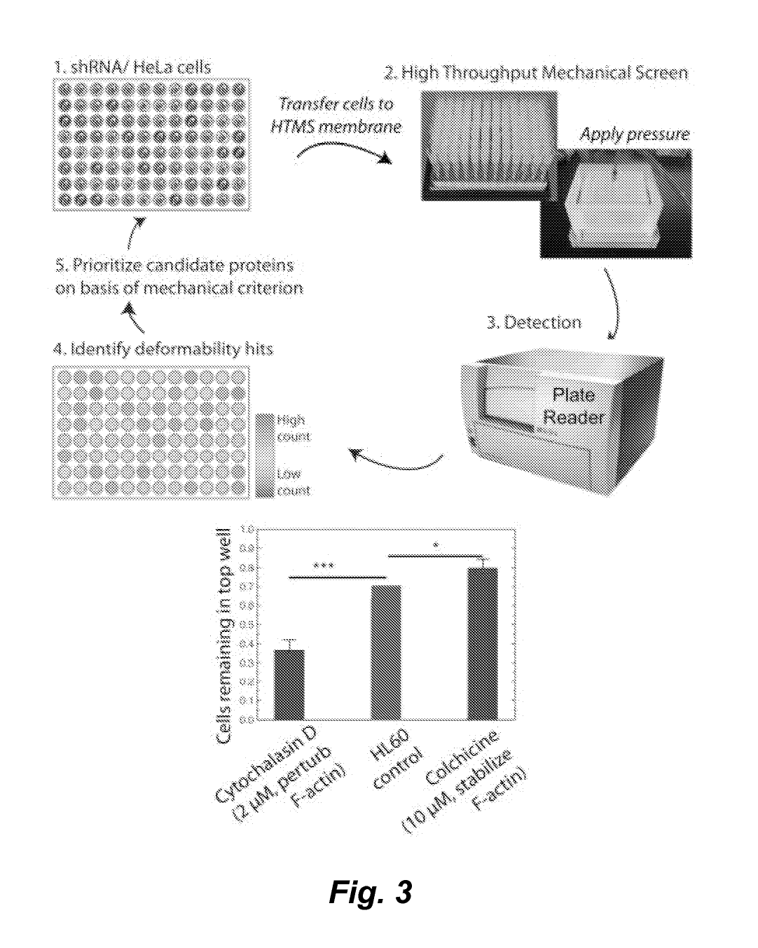

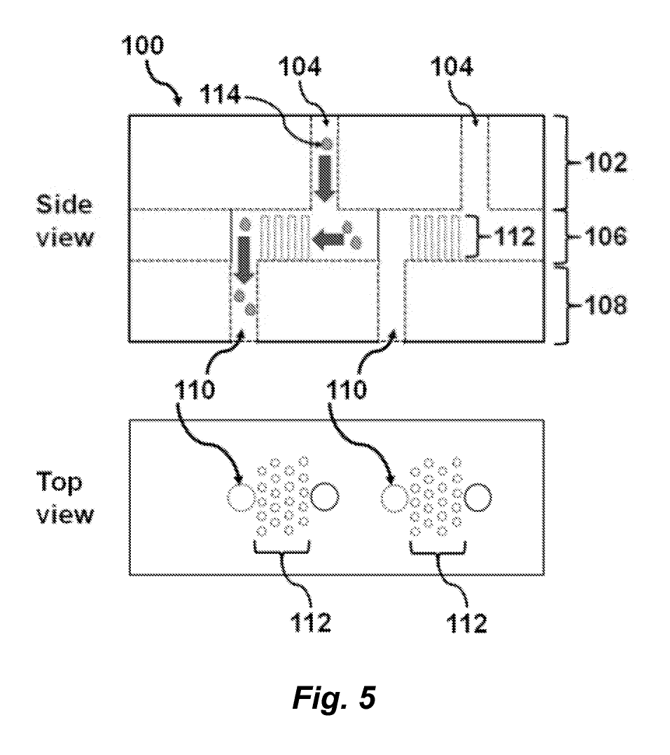

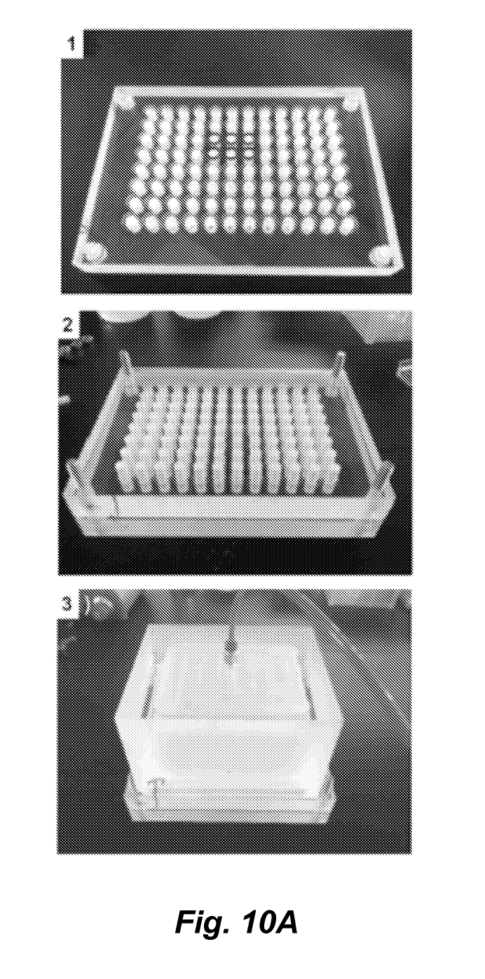

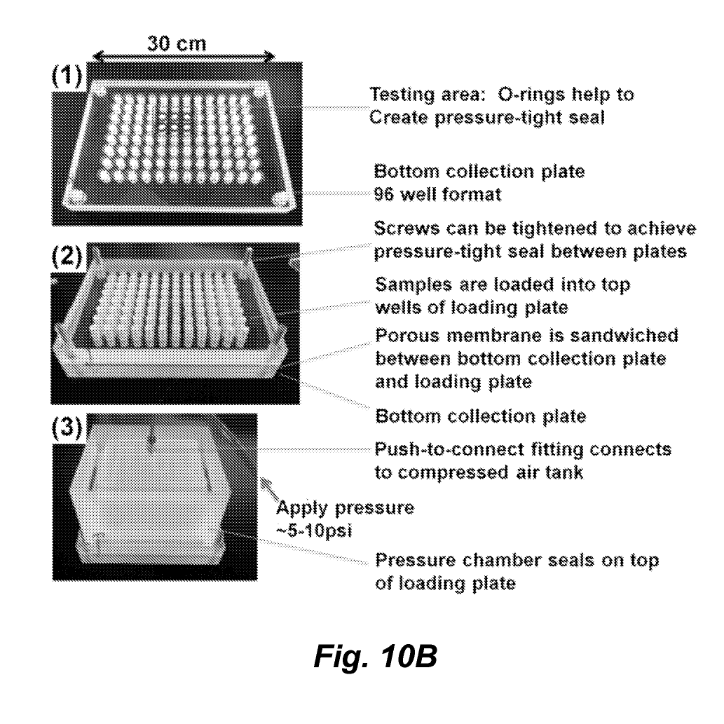

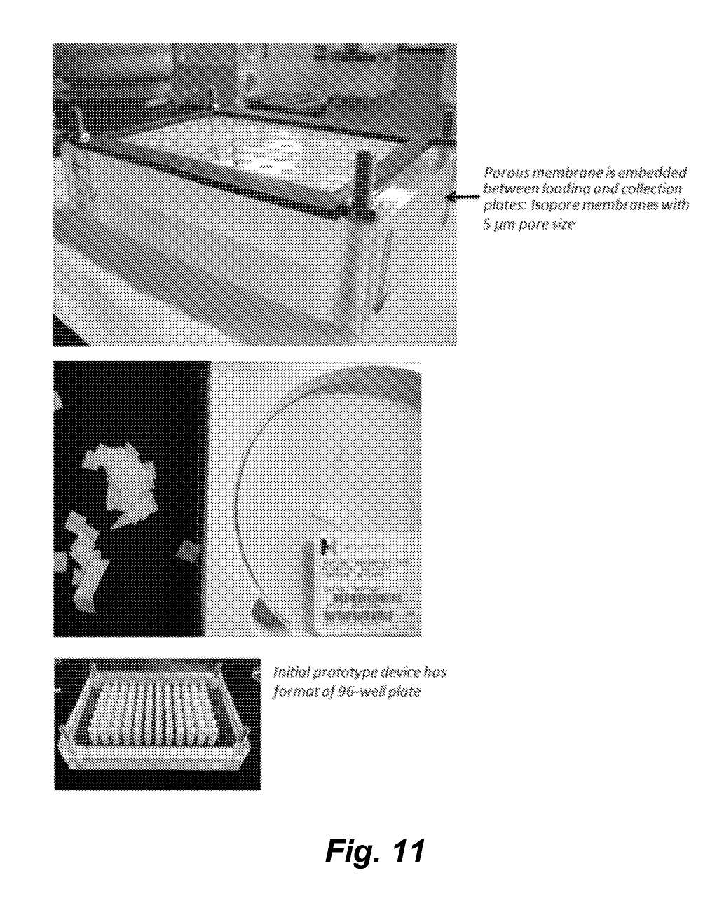

An overview of one embodiment of the HTMS instrumentation and operation is illustrated in FIGS. 2-16. As schematically illustrated in FIGS. 4 and 5, in certain embodiments, the high throughput mechanical screening (HTMS) device 100 comprises a first structure 102 comprising a plurality of sample receiving wells 104, a second structure 108 comprising a plurality of exit ports (or channels or chambers) 110, and a porous structure 106. The porous structure 106 is disposed between the plurality of sample receiving wells 104 and said plurality of exit ports 110 (e.g., between the first structure and the second structure), and in communication (e.g., in fluid communication) with the plurality of sample receiving wells 104 and in communication (e.g., in fluid communication) with the plurality of exit ports (channels or chambers) 110, where the first structure 102, the second structure 108, and the porous structure 106, are sealed or configured such that when sufficient pressure is applied to the sample receiving wells 104 in the first structure 102 the cells or particles 114 migrate through the porous layer into the exit ports. Typically the mean or the median pore size (e.g., channel size) of the porous structure is smaller than the mean or median cross-section of a cell or particle that is to be screened. This results in stiffer (e.g., less deformable) cells or particles failing to pass through the porous structure, while less stiff (e.g., more deformable) cells or particles pass through into the exit ports.

The porous structure 106 can comprise any of a number of porous materials.

For example, in certain embodiments, the porous structure can comprise a porous membrane (e.g., a commercial membrane such as an isopore membrane). In certain embodiments, effective porosity and be created and precisely controlled by fabricating features into the porous structure. Thus, for example, as illustrated in FIG. 4 porosity can created by the introduction of channels 122 in the porous structure, while, as illustrated in FIG. 5 porosity can effectively be created by the introduction of posts (pillars) 112 in the porous structure. In certain embodiments, porosity can be created or controlled by providing channels filled with a particulate material, e.g., of defined particle size or size range where the interstices between the particles create the porosity.

In certain embodiments the pores, channels, interstices, etc. that comprise the porous structure are of a substantially homogeneous size. In some embodiments, the porous structure provide a heterogeneous distribution of pore sizes with pores of different size being located in different zones/regions of the porous structure. In certain embodiments two different regions may provide two different characteristic pore sizes, three different regions may provide three different characteristic pore sizes, and so forth. In certain embodiments, there is a gradient of pore sizes across the wells of a single porous structure (e.g., corresponding to a single multiwell plate). An array of precisely defined pore sizes can easily be generated (using for example soft lithography as described herein). An array of porous structures with a heterogeneous distribution of pore sizes can facilitate: 1)

Screening a complex sample that may consist of subpopulations of cells of different sizes or deformabilities or physical characteristics in a single run; 2) Determining the appropriate pore size to use for a given pressure for a given population, or mixed population of cells that may have different physical characteristics; and/or 3) Subjecting cells to a physiological range of pore sizes in a single run, whereby a “run” is defined as an applied pressure for a defined period of time. Such a heterogeneous array of pore sizes can , for example, recapitulate the dimensions of arterial and venous capillaries.

In certain embodiments the physical properties of the porous structures can be inert, or the surfaces of the porous structure(s) can be modified to promote adhesion or binding. For example, proteins or antibodies can be grafted to the surface; or the surface properties can be altered to render the porous structures hydrophilic or hydrophobic. In such an embodiment, the membranes that contain an array of pores could be used to combinatorially screen cells for both deformability and surface adhesion or interaction with surface molecule

In certain embodiments, as illustrated in FIGS. 4 and 5 the sample receiving chamber(s) 104 can be offset from the exit port(s) 110 permitting the cells and/or particles to pass laterally through the porous structure 106, e.g., as indicated by the block arrows.

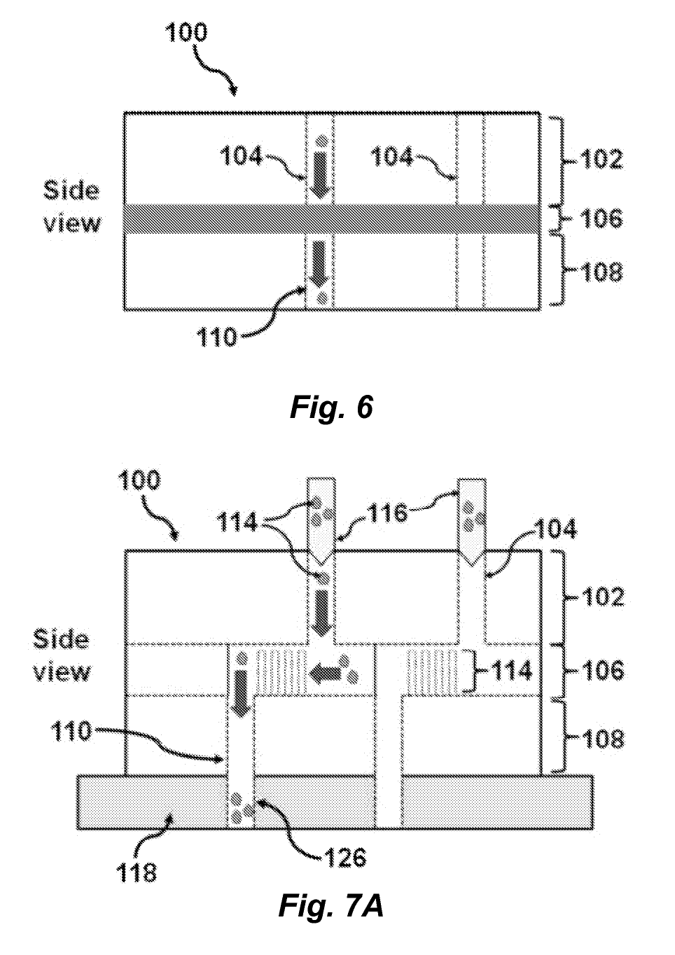

In some embodiments, e.g., as illustrated in FIG. 6 the sample receiving chamber(s) 104 can be aligned with the exit port(s) 110 permitting the cells and/or particles to pass directly through the porous structure 106, (e.g., porous membrane).

It will be recognized, that in various embodiments, the first structure 102, second structure 108, and porous structure 106 can be provided as separate structures (e.g., that are assembled together). However, in certain embodiments, the porous structure 106 and the first structure 102 can be provided as a single structure (e.g., a structure comprising both sample receiving wells 104 and a porous region in communication with those sample wells. In certain embodiments, the porous structure 106 and the second structure 108 can be provided as a single structure (e.g., a structure comprising both sample exit ports 110 and a porous region in communication with those exit ports. In certain embodiments, both, the porous structure 106 and the first structure 102 can be provided as a single structure and the porous structure 106 and the second structure 108 can be provided as a second single structure (e.g., a structure comprising both sample exit ports 110 and a porous region in communication with those exit ports and both structures can be assembled together to make a combined porous structure. In some embodiments, the first structure 102, the porous structure 106, and the second structure 108 can be provided as a single unitary structure (e.g., fabricated using 3-D printing technologies).

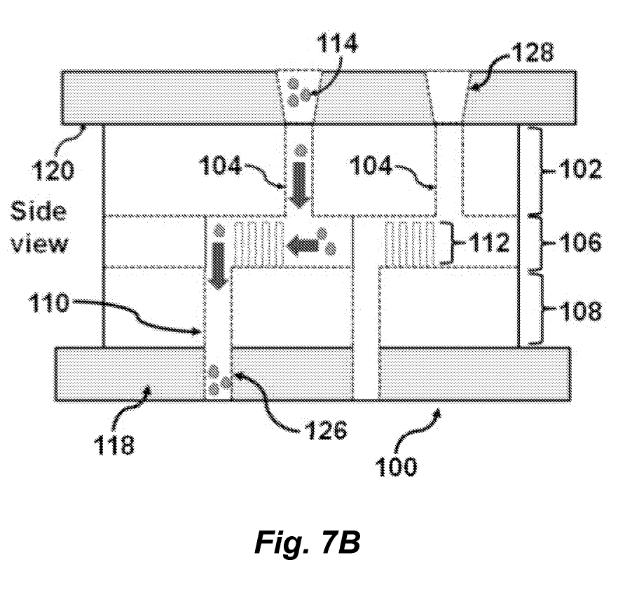

FIGS. 7A and 7B illustrate embodiments of the HTMS device where the device is provided with a receptacle 118 (e.g. a microtiter plate) comprising a plurality of collection wells 126 for collecting cells or particles that pass through the porous structure.

It will be appreciated, that in certain embodiments, the exit ports can be configured with bottoms to form wells out of the ports for collecting cells or particles that pass through the porous structure. While the device is illustrated with a separate collection well 126 for each sample receiving wells, it will be appreciated that in certain embodiments, a single well 126 can be configured to collect cells or particles originating from a plurality of sample receiving wells 104. In some embodiments it is contemplated that a single well 126 can be utilized to collect all the cells or particles.

In some embodiments, no structure is required for collecting the cells or particles. Moreover in some embodiments, the second structure 108 is optional. In such embodiments, the measurement can be made simply by determining the number of cells and/or particles retained in the original sample receiving wells 104 or in the porous structure(s) themselves.

FIG. 7A also illustrates the use of micropippettes 116 to deliver the cells or particles 114 into the sample receiving chambers 104, while FIG. 7B illustrates the use of a sample delivery tray or array of wells (e.g., a loading plate) 120 comprising a plurality of sample delivery wells 128 to deliver the cells or particles 114 into the sample receiving chambers 104.

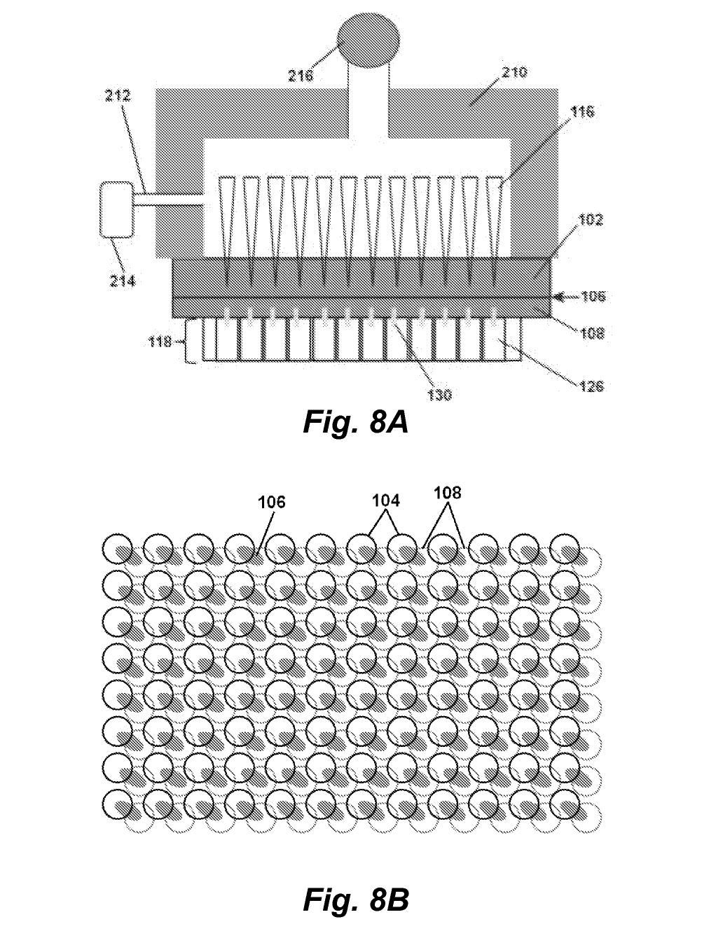

Various configurations of the HTMS device are illustrated in FIGS. 8-12. As shown in FIG. 8A, a cover plate 210 is provided that can seal to the device (e.g. to the first structure 102 permitting all or a subset of the receiving wells 104 in the first structure to be pressurized by a pressurized gas or fluid (see, e.g., FIG. 2).

In various embodiments the porous structure 106 is sealed so that when receiving wells 104 comprising the first structure 102 are pressurized there is little or no leakage around the porous layer and preferably no substantial leakage out the edges of the porous layer. In short, in various embodiments the porous layer is sealed so that pressure applied to the wells in the first structure 102 acts to drive particles and/or cells through the porous structure 108 into exit ports 110, and optionally into corresponding wells 126 in a sample collection plate 118. Where a second sample collection plate is omitted the particles and/or cells can simply pass into a waste receptacle or general collection chamber.

In some embodiments, the cover plate 210 can be equipped with or provide a connection to a pressure gage 216 to monitor the applied pressure. The cover plate can also be provided with a connection to a pressure source 214 (e.g., a gas tank, or other pressure source).

As schematically illustrated in the embodiment shown in FIG. 8A, an array of pipette tips (e.g., 96 tip array) is inserted into the first structure 102 (e.g., a top PDMS layer). Any other method that provides sample delivering chambers or structures in fluid communication with the sample receiving chambers can also be used to deliver cells, particles, and/or reagents into the sample receiving wells 104. In the example, illustrated in FIG. 8A and 8B, porous structures can patterned onto the bottom side of this first structure using, for example hard etching methods, or soft lithography. A bottom layer of PDMS can contain the exit port(s) 110 and can be covalently bonded to the top structure of PDMS. IN the illustrated embodiments a standard microtiter plate (e.g., a 96-well plate) can be used to collect the cells or particles. The number of collected cells is then counted. In other embodiments, the number of passaged cells can be determined by counting the remaining cells in the sample delivering chamber; or by counting the number of cells trapped in the porous structure. As clearly illustrated in the top view (FIG. 8B), the porous structure 106 resides at the interface between the first structure 102 and the second structure 108.

In certain embodiments, small pieces of tubing or tubular structures 130 are optionaly inserted into the exit port(s) 110 to promote the exit of fluids from the structure. This may help to prevent the fluid from wetting the underside of the porous structure and promotes the delivery of cells or particles into the collection chamber.

In certain embodiments the porous structure 108 can be permanently affixed in the device, while in other embodiments, the porous structure can be removed and replaced. To induce deformation in the cells or particles, a structure is selected where the pore sizes are smaller than the dimensions of the cell or particle. In certain illustrative but non-limiting embodiments the average Rₚₒᵣₑ/R_{cell} is less than about 0.9, more preferably less than about 0.8, still more preferably less than about 0.7 or 0.6. In certain embodiments, Rₚₒᵣₑ/R_{cell} ˜0.5. For cells that range from 10-30 μm, to provide such conditions porous structures with pores (or channels, and the like) that have a diameter that ranges from about 5 μm to about 15 μm are desirable. Microgels and liposomes can be fabricated to have similar dimensions.

Device Fabrication

The HTMS devices described herein can be fabricated using any of a number of methods well known to those of skill in the art and they can be fabricated out of any of a number of suitable materials. Illustrative materials include, but are not limited to plastic, glass, ceramic, and various elastomeric materials, particularly elastomeric materials used in soft lithography.

There are many formats, materials, and size scales for constructing HTMS systems described herein. In certain embodiments the devices described herein are made of PDMS (or other polymers) fabricated using a technique called “soft lithography”. PDMS is an attractive material for a variety of reasons including, but not limited to: (i) low cost; (ii) optical transparency; (iii) ease of molding; (iv) elastomeric character; (v) surface chemistry of oxidized PDMS can be controlled using conventional siloxane chemistry; (vi) compatible with cell culture (non-toxic, gas permeable). Soft lithographic rapid prototyping can be employed to fabricate the HTMS devices.

One particularly useful property of soft lithography materials such as PDMS and the like is that their surface can be chemically modified in order to obtain the interfacial properties of interest (see, e.g., Makamba et al. (2003) Electrophoresis, 24(21): 3607-3619). On illustrative method of covalently functionalizing PDMS is to expose it to an oxygen plasma, whereby surface Si—CH₃ groups along the PDMS backbone are transformed into Si—OH groups by the reactive oxygen species in the plasma. These silanol surfaces are easily transformed with alkoxysilanes to yawed many different chemistries (see, e.g., Silicon Compounds: Silanes and Silicones, Gelest, Inc., Morrisville, Pa., 2004; p. 560; Hermanson et al. (1992) Immobilized affinity ligand techniques, Academic Press, San Diego, Calif. 1992). Using such surface modification methods, in certain embodiments, proteins, nucleic acids, or antibodies can be grafted to the porous structure surface, or the surface properties can be altered to render the porous structures hydrophilic or hydrophobic. In such an embodiment, the HTMS devices can be used to screen cells for both deformability and surface adhesion or interaction with surface molecule.

One illustrative version of soft lithographic methods involves preparing a master (mold) (e.g., an SU-8 master) to form the microchannel system, pouring a pre-polymer onto the master and curing it to form a cured patterned replica (e.g., PDMS polymer replica), removing the replica from the master and trimming and punching tubing inlets as required, optionally exposing the polymer to a plasma (e.g., to an O₂ plasma) and optionally bonding the polymer to a substrate (e.g., a glass substrate).

The master mold is typically a micromachined mold. Molds can be patterned by any of a number of methods known to those of skill in the in the electronics and micromachining industry. Such methods include, but are not limited to wet etching, electron-beam vacuum deposition, photolithography, plasma enhanced chemical vapor deposition (PECVD), molecular beam epitaxy, reactive ion etching (RIE), and/or chemically assisted ion beam milling (CAIBM techniques), and the like (see, e.g., (1997) The Handbook of Microlithography, Micromachining, and Microfabrication, Soc. Photo-Optical Instru. Engineer, Bard & Faulkner (1997) Fundamentals of Microfabrication, and the like).

Another illustrative micromachining method uses a high-resolution transparency film as a contact mask for a thick photoresist layer. Multilayer soft lithography improves on this approach by combining soft lithography with the capability to bond multiple patterned layers of elastomer. Basically, after separate curing of the layers, an upper layer is removed from its mold and placed on top of the lower layer, where it forms a hermetic seal. Further curing causes the two layers to irreversibly bond. This process creates a monolithic three-dimensionally patterned structure composed entirely of elastomer. Additional layers are added by simply repeating the process. The ease of producing multilayers makes it possible to have multiple layers of fluidics, a difficult task with conventional micromachining

While the fabrication of the present devices is described with respect to the use of PDMS as a soft lithography material, it will be recognized that, in various embodiments, numerous other materials can be substituted for, or used in conjunction with PDMS. Illustrative materials include, but are not limited to polyolefin plastomers (POPs), perfluoropolyethylene (PFPE), polyurethane, polyimides, and cross-linked NOVOLAC® (phenol formaldehyde polymer) resins.

In various embodiments, single-layer or multi-layer PDMS (or other material) devices are contemplated. In illustrative approach, an array of features comprising a porous structure is designed in a CAD program. This design is converted into a transparency by a high-resolution printer; this transparency is used as a mask in photolithography to create a master in positive relief photoresist. PDMS cast against the master yields a polymeric replica containing the features. The surface of this replica, and that of a corresponding featured or a flat slab of PDMS, can be oxidized in an oxygen plasma. These oxidized surfaces seal tightly and irreversibly when brought into conformal contact. Oxidized PDMS also seals irreversibly to other materials used in microfluidic systems, such as glass, silicon, silicon oxide, and oxidized polystyrene. Oxidation of the PDMS has the additional advantage that it yields channels whose walls are negatively charged when in contact with neutral and basic aqueous solutions; these channels can be filled easily with liquids with high surface energies (especially water).

The fabrication methods described herein are illustrative and not limiting. Using the teachings provided herein, numerous other photolithographic and/or micromachining techniques can be used to fabricate the devices described herein. The micromachining and soft lithography methods described above, as well as many others, are well known to those of skill in the art (see, e.g., Choudhury (1997) The Handbook of Microlithography, Micromachining, and Microfabrication, Soc. Photo-Optical Instru. Engineer, Bard & Faulkner (1997) Fundamentals of Microfabrication; McDonald et al. (2000) Electrophoresis, 21(1): 27-40).

The first structure and second structures are easily fabricated using soft lithography or other techniques, e.g., as described herein. In some embodiments, the porous structure can be a commercially available porous structure. Polycarbonate (and other) membranes with such pore sizes are commercially available (Millipore), and can be exploited for use as the porous structure in the devices described herein . Illustrative suitable porous membranes are shown in Table 1.

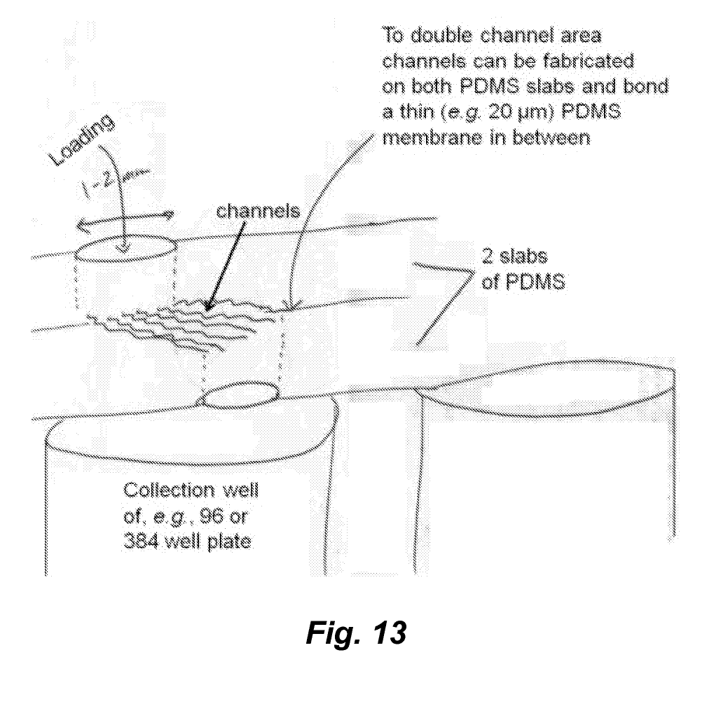

In certain embodiments, however, the porous structures are readily fabricated to desired geometry and porosity using well known lithographic methods including, but not limited to soft lithography. For example, in one embodiments, the bottom of the first structure 102 and/or the top of the second structure 108 are fabricated with posts (pillars) at a particular spacing and/or channels and/or pores of particular desired dimensions (e.g., selected to provide a desired effective porosity). When the first structure 102 is bonded to the second structure 108, the interface forms the porous structure 106 (see, e.g., FIG. 13).

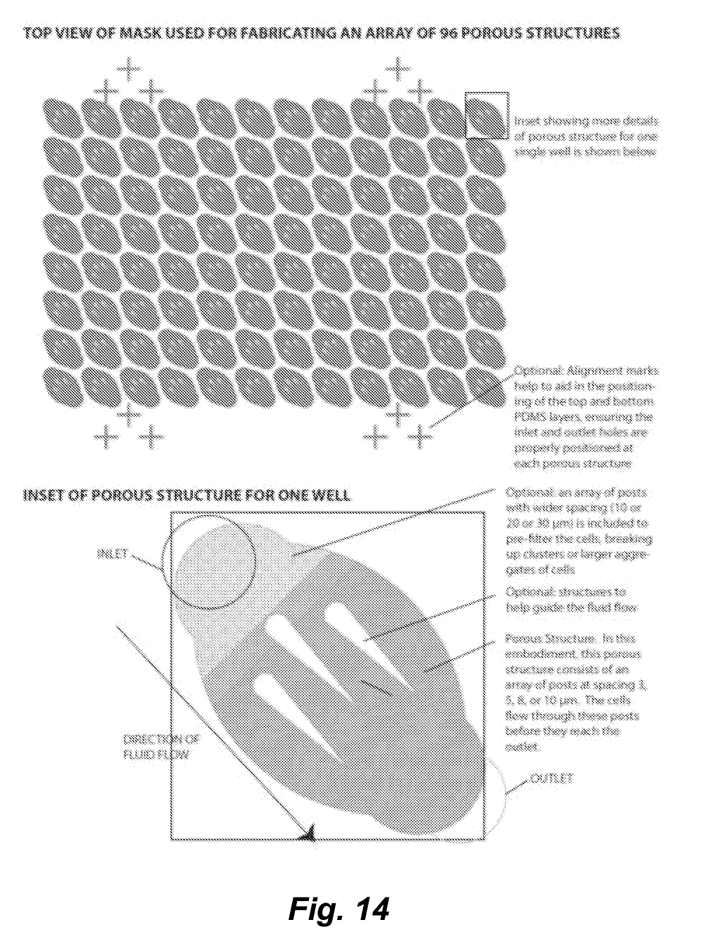

FIG. 14 illustrates a mask used to fabricate an array of 96 porous structures and it will be recognized that the number can easily be scaled up and down.

As shown in FIG. 14, in some embodiments, an optional array of posts with “wide” spacing (e.g., (10 μm, or 20 μm, or 30 μm, etc.) can be included to pre-filter the cells breaking up clusters or larger aggregates of cells. As shown, features to help guide fluid flow are also readily incorporated. In the embodiment illustrated in FIG. 14, the porous structure comprises an array of posts at spacing 3, 5, 8, or 10 μm. The cells flow through these posts before they reach the outlet.

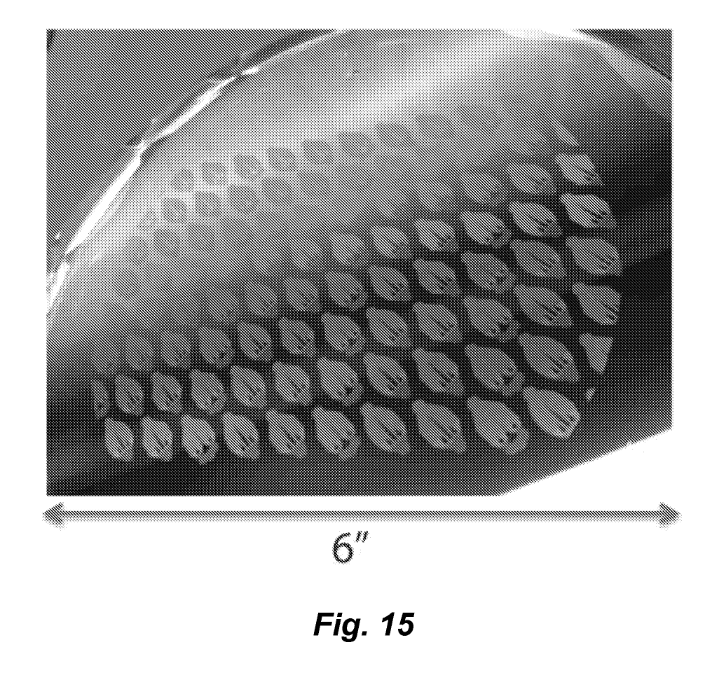

FIG. 15 illustrates the fabrication of “posts” for a porous structure. Arrays of posts are fabricated using soft lithography on a 6″ silicon wafer. This provides a master or mold for the polydimethysiloxane (PDMS).

In various embodiments the porous structure is fabricated so it can be incorporated into a standard multi-well plate format (e.g., 6, 12, 24, 48, 96, 384, 1536-well) plate. For example, in one illustrative embodiment, laser cutting can be used to create a 96-well insert with 5 μm pores drilled into it.

Soft lithography can also be used to custom-fabricate passage regions with varying pore sizes and geometries. Given that the time required for a cell and its nucleus to passage through a pore depends largely on its elastic modulus, it is believed that by increasing the pore length, as well as the number of constrictions along the path length, differences in passage time between cells with altered mechanical properties can be amplified.

The concept underlying this design is based on similar principles as electrophoresis, where the passage or retention time of biomolecules is set by their physical properties as well as the gel mesh size and applied force; by tuning the pore size and path length relative to the deformability of cells, the detection resolution in the methods and devices described herein can be set.

“Tuned” porous “membranes” can be produce for example by fabricating microfluidic channels with varying degrees of tortuosity and imaging cells as they passage through the channels using a high speed camera (Phantom Miro ex4, Vision Research). The optimized channel designs identified thereby can be used to fabricate a porous PDMS membrane with controlled spatial distribution of channels so the membrane interfaces directly with, for example, a 384-well plate to facilitate sample loading and collection. In this regard, it is noted that many existing shRNA and drug libraries are in 384-well format. The ability to use this improved ‘High Resolution’-HTMS (HR-HTMS) methodology with existing plate- and liquid-handling robots, shRNA libraries, as well as conventional flow cytometers facilitates the rapid use of the HTMS systems described herein.

Operation of the Device

To first order, the relevant parameters that affect the number of passaged cells can be represented in Equation I:

$$\frac{{N}_{passaged}}{{N}_{loaded}} = f ( Δ P , t , \frac{{R}_{cell}}{{R}_{pore}} , {E}_{cell} , {η}_{cell} ) \qquad I$$

where N_{passaged} is the number of passaged cells, N_{loaded} is the number of loaded cells, AP is the applied pressure, t is the time of applied pressure, R_{cell} is the radius of the cell, ore is the radius of the pore, E_{cell} is the elastic modulus of the cell , and η_{cell} is the viscosity of the cell. The same parameters may also apply to particles. Similarly, the time for deformation can be given as shown in equation II:

$${τ}_{deform} = f \underbrace{( Δ P}_{\begin{matrix}Applied \\ pressure\end{matrix}} , \underbrace{\frac{{R}_{cell}}{{R}_{pore}}}_{\begin{matrix}Cell \\ Size\end{matrix}} , \underbrace{{E}_{cell} , {η}_{cell} )}_{\begin{matrix}Cell Mechanical \\ Properties\end{matrix}} \qquad II$$

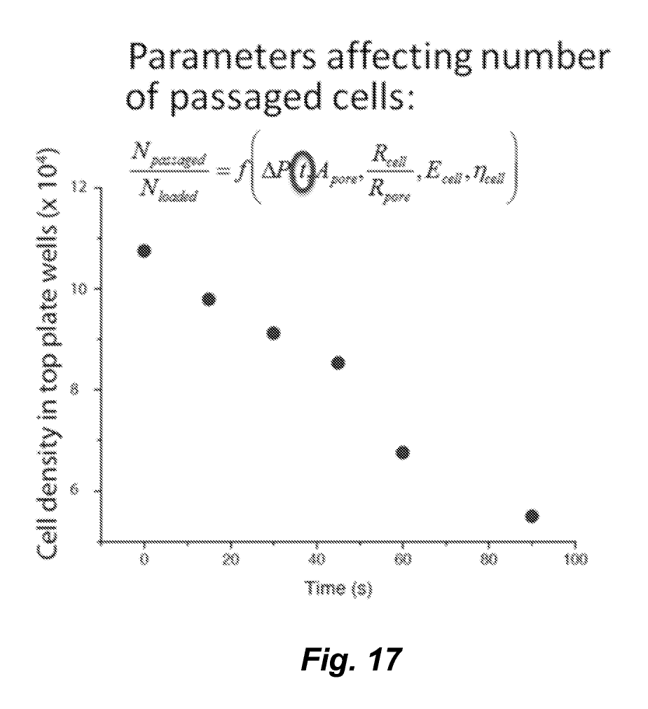

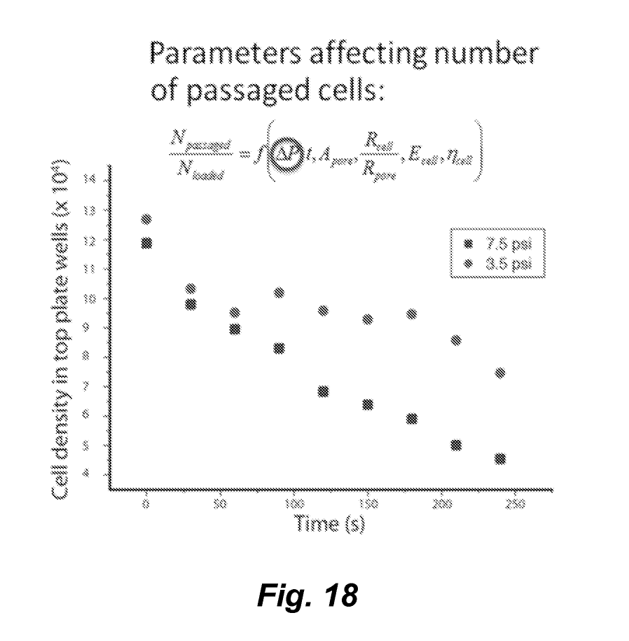

The effect of time (passage of cells as a function of time) and the effect of pressure (passage of cells as a function of pressure is shown in FIGS. 17 and 18, respectively. Using a combination of experiments and modeling, the theoretical description of factors that affect deformation time can be further refined. Below we describe the design and use of an illustrative prototype.

In use, cells, particles or other materials are deposited into various wells in the first “sample” plate. In certain embodiments this “plating” process can be made more efficient using a multichannel pipettor. The optimal loading density of cells can be established iteratively in conjunction with the detection method. Certain preliminary studies suggest that a loading density of approximately 10⁴-10⁶ cells/mL is suitable (see, e.g., FIG. 24). In certain embodiments optimal loading density of cells established iteratively with detection resulted in a loading density of ˜10⁶ cells/mL is given the estimated lower detection limit of 10² cells/mL using a flow cytometer. Using standard multi-well plates (e.g., a 96 well plate, a 384 well plate, a 1536 well plate, etc.) as the second sample “collection” plate enables our device to interface with existing high throughput robotics that are capable of parallelization of liquid handling for multiwell plates.

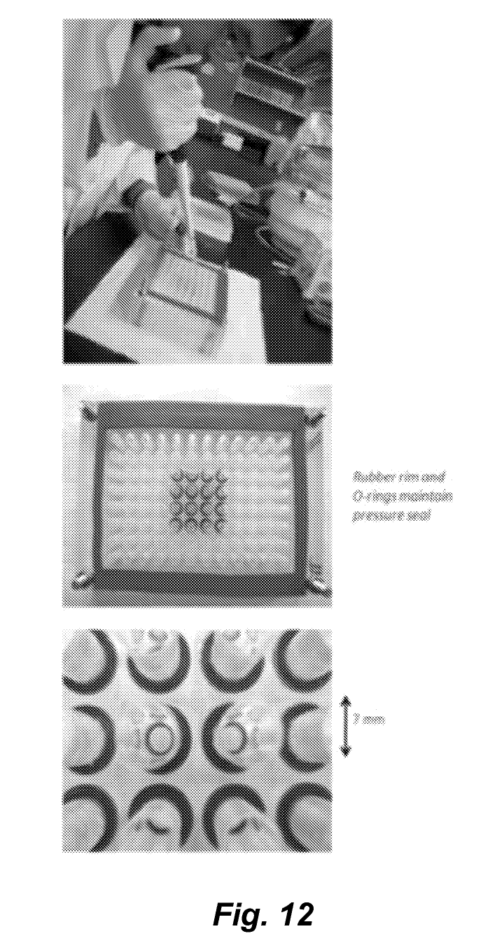

The cells or particles loaded into different sample wells can be different cells or particles, and/or can be cells or particles that have been pre-treated (e.g., transfected with various genes, or inhibitor nucleic acids, or contacted with particular reagents (e.g., various putative pharmaceuticals) before being loaded into the sample wells (see, e.g., FIG. 12). In certain embodiments the cells and/or particles in different wells are the same and different test reagents (e.g., various inhibitory nucleic acids, various putrative pharmaceuticals, etc.) are loaded into different wells. In certain embodiments various wells can be loaded with no test reagents to provide negative controls, or with various reagents known to have a particular activity/effect to provide positive controls.

In certain embodiments the test agents are members of a library (e.g., an RNAi library, a pharmaceutical library, a peptide library, and the like) that is to be screened for activity that alters the mechanical properties of the cells or particles.

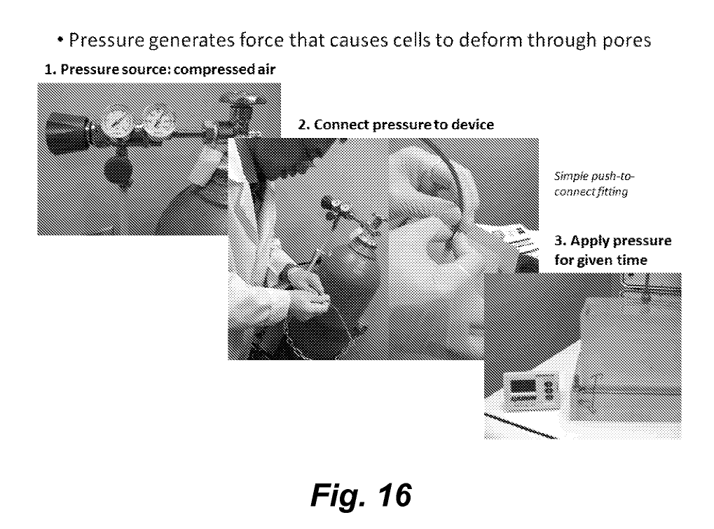

Once the first plate of the device is loaded, the lid is placed on top to create a pressure-tight seal. In certain embodiments tubing can be connected via easy-to-use push-to-connect fittings (McMaster Carr) (see, e.g., FIG. 16). In some embodiments, to deliver gas pressure, an electro-pneumatic valve can be placed in the gas line, enabling precise control of applied pressure for sub-second periods of time by a convenient and friendly computer-controlled user interface. A compressed air tank provides a convenient pressure source: for example, 5% CO₂ (or N₂ or He₂, argon, or other gases or gas mixtures) outfitted with a pressure gauge (e.g., 0-30 psi, with 0.5 psi resolution) (Airgas). In certain embodiments 5% CO₂ is preferred as this gas mixture is standard for the culture of many mammalian cell types.

Pressure need not be delivered by a gas. In certain embodiments a fluid pressure system is also contemplated. In certain embodiments the fluid used to pressurize the wells can comprise a suitable buffer or culture medium. The fluid can be pressurized using standard methods well known to those of skill in the art. For example, in certain embodiments a fluid pump (e.g., a syringe pump) can be used. In certain embodiments the fluid can be pressurized using a pressurized gas source, and so forth.

The HTMS is pressurized for a length of time sufficient to permit various cells to mover from the wells in the first plate, through the porous layer, into wells in the second plate. The length of time is a function, in part, of the mechanical properties of the cells, the nature of the porous medium and the applied pressure. In certain embodiments the HTMS is pressurized for a time ranging from about 1, or about 2, or about 3, or about 4, or about 5, or about 6, or about 7, or about 8, or about 9, or about 10 seconds up to about 20 s, or up to about 25 s, or up to about 30 s, or up to about 40 s, or up to about 50 s, or up to about 60 s, or up to about 90 s, or up to about 120 s, or up to about 180 s, or up to about 240 s, or up to about 300 s, or up to about 400 s, or up to about 500 s, or up to about 600 s, or up to about 700 s, or up to about 800 s, or up to about 1000 s, or longer. These pressurization times are illustrative and not limiting and can vary depending on the applied pressure.

In certain embodiments, the applied pressure ranges from about 0.1 psi up to about 30 psi, or up to about 25 psi, or up to about 20 psi, or up to about 15 psi, or up to about 10 psi. In certain embodiments the applied pressure is about 0.2 psi, or about 0.3 psi, or about 0.4 psi, or about 0.5 psi, or about 0.6 psi, or about 0.7 psi, or about 0.8 psi, or about 0.9 psi, or about 1.0 psi, or about 1.1 psi, or about 1.2 psi, or about 1.3 psi, or about 1.4 psi, or about 1.5 psi, or about 1.6 psi, or about 1.7 psi, or about 1.8 psi, or about 1.9 psi, or about 2 psi, or about 2.1 psi, or about 2.2 psi, or about 2.3 psi, or about 2.4 psi, or about 2.5 psi.

The number of cells that have passaged through the porous filter from each well of interest is then determined. Any of a number of well-known methods for counting cells can be used. For example, in certain embodiments, to efficiently count cell numbers in the individual wells (e.g., of a 96-well plate) a conventional flow cytometer with high throughput capabilities can be utilized. Using light scattered from individual cells, the number of passaged cells per unit volume is determined by counting particle number per volume in each individual well. An entire 96-well plate can be scanned within a short period of time. For example, using the LSRII flow cytometer (BD), an entire 96-well plate can be scanned within 15 minutes, while sampling a minimum of 2 μL volume fixed flow rate from each individual well. Flow cytometers are readily available, e.g., in core biological research facilities. Moreover, benchtop flow cytometers for high throughput cell and particle detection can be purchased for less than $50,000 (e.g. Guava, Millipore). Flow cytometry also enables high information content screening, as expression levels of particular protein(s) or gene(s) can also be determined using fluorescence detection, and correlated with deformability. In certain embodiments a plate reader could be used for obtaining a count of cells stained with some fluorophore enabling fluorescence detection.

More broadly, one can just count the cells that passage through the porous structure. This can be achieved, for example, by electrical detection, or light scattering, or any other method that is used to detect the presence of cells. Depending on the detection method, a second plurality of wells may or may not be used. For example, real-time detection may rely simply on altered capacitance due to flow of cells across a charged porous membrane, but other methods for detecting cells may also be used.

More sophisticated detection methods enable more accurate detection of cells passaging per unit time. For example, interfacing the membrane with electrodes, the number of cells passaging through pores could be quantified using resistance changes.

In certain embodiments the HTMS device can be calibrated using, for example, a combination of synthetic gel particles that are fabricated to have distinct elastic moduli, and/or using oil-in-water droplets that have a range of viscosities, and/or using previously characterized cell systems whose mechanical properties are altered by drugs and genetic methods (see, e.g., Rosenbluth et al. (2008) Lab Chip. 8: 1062-1070; Hanss (1983) Biorheology. 20: 199-211; Moessmer and Meiselman (1990) Biorheology 27: 829-848).

With respect to the use of gel particles as a calibration standard, it is noted that polyacrylamide particles can readily be fabricated with a range of crosslinker densities, and thus elastic moduli (E˜1-55 kPa) (see, e.g., Yeung et al. (2005) Cell Motil Cytoskeleton, 60: 24-34; Wyss et al. (2010) Soft Matter, 6: 4550-4555). Such particles enable calibration of the HR-HTMS to distinguish particles based on their elastic modulus. Moreover, the gel particles each have the same composition, and thus stiffness; this helps to distinguish sample heterogeneity and measurement variability. To test the effect of viscosity, drops of oil-in-water emulsions can be generated using silicone oils that have a range of kinematic viscosities (υ=1-1000 cSt, PDMS 200, Dow Corning). The number of passaged droplets can be counted by imaging, and can be used to characterize the effect of a range of internal viscosities.

Certain embodiments showing overall device architecture and experiment workflow are shown in FIGS. 2-16. As illustrated therein, the entire HTMS setup can be designed for ease of use. For example, in one illustrative mode of operation, the porous membrane insert is removed from its sterile package and placed into a standard 96-well plate. Individual cell samples are then deposited into the wells of the insert. The custom, pressure-sealable lid is place on top; and the lid is connected to a source of compressed air by inserting tubing into the user-friendly, push-to-click fitting. Alternatively, cell samples may be loaded into pipette tips that are directly inserted into the holes of the custom PDMS membrane. Using computer control, pressure is applied to the device (e.g., to the first plate) for a given time. Then the pressure seal is disconnected and the plate is placed into a flow cytometer for efficient counting of cells in each individual well. Using standard flow cytometer software, a heat map of cell densities across the wells is plotted, enabling facile determination of cell numbers, and thereby linking cell deformability/ mechanical phenotype to the original cell genotype/environmental conditions. Another detection method such as a plate reader could also be used to quantify the number of cells per well.

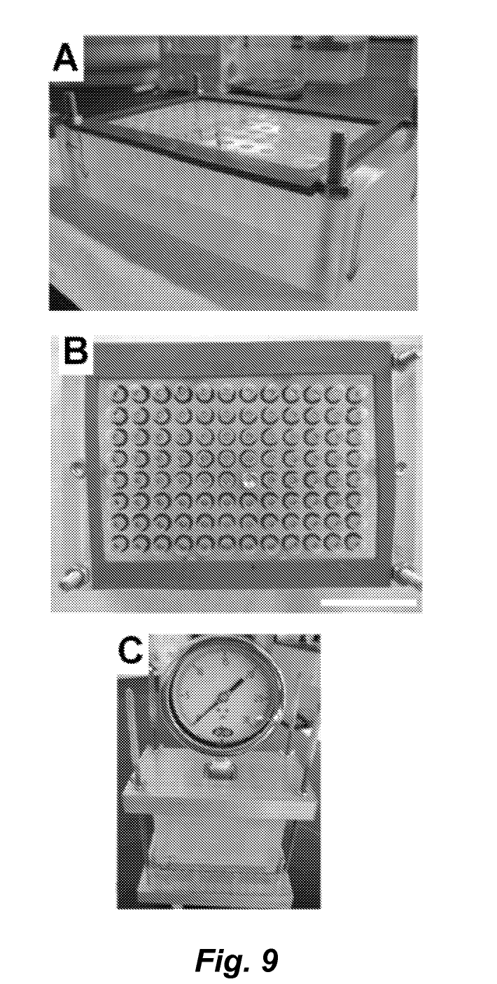

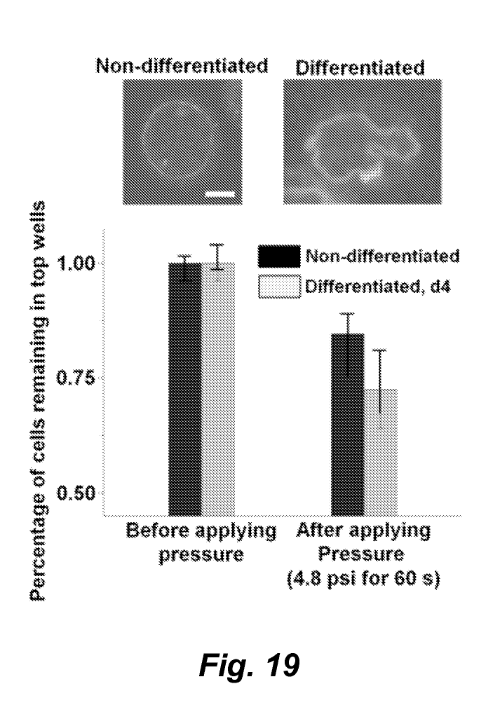

A prototype device has been fabricated (see, e.g. FIGS. 9-11). Initial testing with HL60 cells shows evidence that the device is working as designed. The number of passaged of cells increases with the amount of time of applied pressure (FIG. 18) and the number of passaged cells per unit time increases with applied pressure (FIG. 19).

The foregoing embodiments are intended to be illustrative and not limiting. Using the teachings provided herein, numerous other configurations of first plate, porous layer, and second plate, means of pressurization, and detection of cells that pass through the porous layer will be available to one of skill in the art.

### EXAMPLES

The following examples are offered to illustrate, but not to limit the claimed invention.

### Example 1

### Probing the Nucleus by Flow Through Constricted Microfluidic Channels

To dissect the contributions of nuclear physical properties to whole cell deformability, human promyelocytic leukemia (HL60) cells provide a convenient model system. These cells can be readily differentiated into neutrophil-type cells in vitro. During this process of granulopoiesis, the cell nucleus undergoes major changes in morphology as it transitions from an ovoid to an irregular, multi-lobed structure. Not surprisingly, the differentiated cells transit more rapidly through constricted channels as compared to the non-differentiated control cells.

In addition to these induced changes in nuclear morphology, cytoskeletal protein expression is also altered. Levels of actin increase during differentiation, and cortical actin is implicated in the resistance of these cells to out-of-plane deformations. To determine the contributions of actin to cellular deformation, cells were treated with a drug (cytochalasin B) to perturb their actin network. These experiments revealed no detectable difference between treated and non-treated controls, further suggesting that the nucleus is the main arbiter of cell physical properties when cells deform through sufficiently narrow constrictions. One determinant of nucleus shape stability is the intermediate filament type protein, lamin A. This protein is a major constituent of the meshwork underlying the nuclear envelope, and its expression levels are directly linked to nuclear mechanical properties.

This is consistent with the dependence of reconstituted cytoskeletal protein network mechanical properties on protein density. Interestingly, a major decrease in lamin A expression levels accompanies the altered nuclear morphology that develops during granulopoiesis. To study the effect of lamin A levels on nuclear morphology and whole cell deformability, we genetically modified HL60 cells by retroviral transduction to constitutively express lamin A. After verifying correct localization of the protein by immunofluorescence, and sorting the cells to enrich the population for cells with increased lamin A expression levels, we showed that nuclear shape during granulopoiesis is sensitive to lamin A levels. The high lamin A expressing cells also exhibit a striking change in mechanical properties: we observe a ten-fold increase in median deformation time compared to the mock-modified control cells. These findings are consistent with our studies of LMNA-null mouse embryo fibroblast (MEF) cells that lack lamin A, and show decreased deformation times compared to the wild type controls.

Taken together these results show that the nucleus is the rate-limiting step in whole cell passage through a narrow constriction. More broadly, these findings validate the applicability of this microfluidics methodology to probe cell nucleus mechanical properties.

### Example 2

### Device Fabrication and Testing

An initial prototype device (see, e.g., FIGS. 9-16) was machined out of polymethylmethacrylate (McMaster Carr) in the format of a 96-well plate containing: a porous membrane sandwiched between a bottom, multiwell collection plate, and a top loading plate. A pressure chamber seals the device and interfaces with a compressed air tank (see, e.g., FIG. 17).

In the initial prototype device, we use track-etched, polycarbonate membranes with 5 μm pore size (Millipore). To minimize adhesion, surface treatment with 10 mg/mL bovine serum albumin was performed prior to the experiment. To load the device, cells were deposited on top of the porous membrane in each individual well. The optimal loading density of cells was established iteratively with detection: a loading density of ˜10⁶ cells/mL was optimal given the estimated lower detection limit of 10² cells/mL using a flow cytometer.

Once the device was loaded, the pressure chamber lid was placed on top to create a pressure-tight seal, and tubing was connected to a tank of compressed air with 5% CO₂ that was outfitted with a pressure gauge (0-30 psi, 0.2 psi resolution, Airgas). An electro-pneumatic regulator (0-20 psi output, Omega) enabled precise control of the magnitude and duration of applied pressure for sub-second periods of time via control through LabVIEW. Pressures between 5-10 psi were estimated to generate ˜kPa-scale stresses required to deform cells with elastic moduli in the kPa range.

After applying pressure to induce deformation of cells through the pores, the number of passaged cells was counted. We used a flow cytometer to count cells in the individual wells of a 96-well plate using scattered light (forward versus side scatter) as a signal (LSRII flow cytometer, BD Biosciences). An entire 96-well plate was counted within 15 minutes.

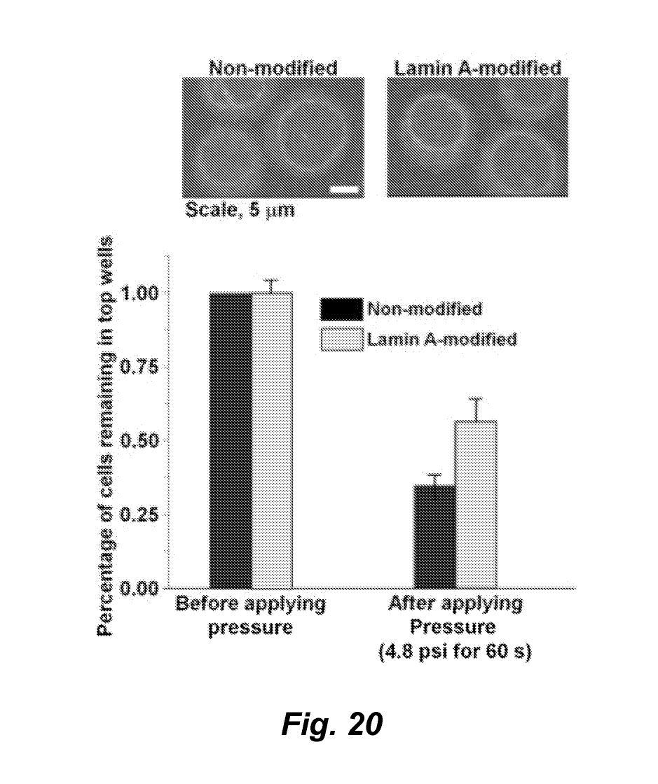

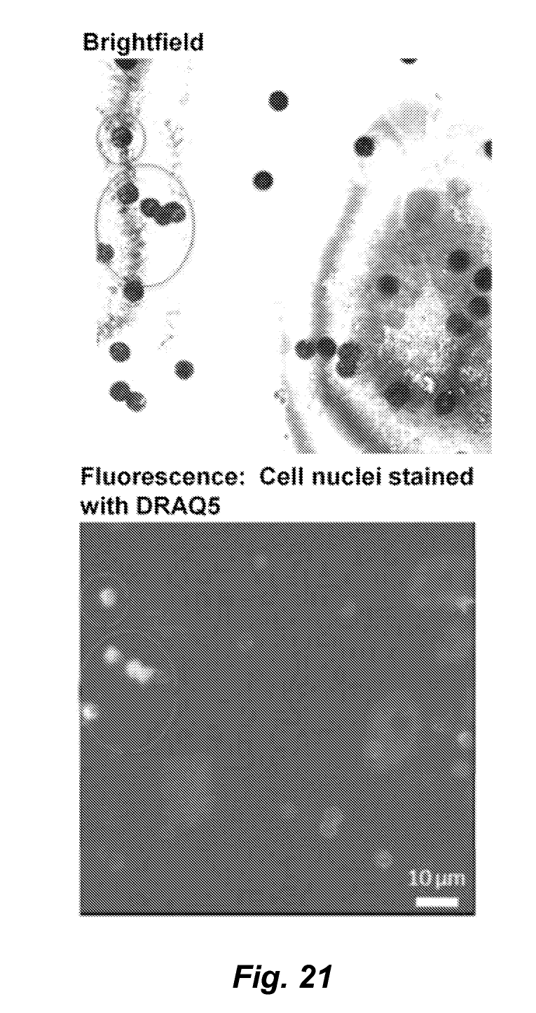

Proof-of-concept experiments demonstrate the feasibility of HTMS methodology for probing differences in nuclear physical properties. We tested HL60 cells in their undifferentiated versus differentiated state, confirming previous results showing these cells have distinct differences in their passage times, as probed by microfluidic deformation and bulk filtration. When screened by HTMS, we observed a significant difference in the number of cells passaged, reflecting differences in the deformability of these two cell populations (see, e.g., FIG. 19). We then conducted a pilot mechanical screen using the genetically modified HL60 cell line, probing lamin A-overexpressing HL60 cells versus the mock-modified controls: using microfluidic deformation, we observed a ten-fold difference in median deformation times between these two populations, with the lamin-A modified cells requiring longer times to passage through narrow constrictions. When screening with HTMS, we observed a similar trend in cell deformability, with a smaller fraction of lamin A -modified cells passaging through compared to the mock-modified controls (FIG. 20).

### Example 3

### Screens With A Spectrum Of Genetic Variants

A test of HR-HTMS performance for the RNAi mechanical screen is the ability to distinguish between genetically modified cells that are expressing different levels of particular proteins. To establish the dynamic range, specificity, and statistical power of HR-HTMS, we will perform an initial pilot screen with mock-modified HL60 cells versus lamin A-modified cells using our custom-fabricated PDMS membranes. With the initial prototype HTMS device, we previously showed a decreased passage number of these cells compared to the mock-modified controls (FIG. 20). Ultimately cell variants in a proteome-wide screen may exhibit more subtle changes in mechanical phenotype than the dramatic effects of lamin A knockout or overexpression. For example, emerin is a nuclear envelope protein that is implicated in Emery-Dreyfuss muscular dystrophy. Our work has shown that emerin-deficient MEF cells have altered nuclear mechanical properties when probed by confocal imaged micro-deformation, and exhibit greater fluctuations in shape over time compared to wild type cells. However, the shape abnormalities of emerin-deficient cells are less extreme than the lamin A-null phenotype30, so knockdown of emerin provides an intermediate mechanical phenotype for a reference screening system.

One model system of choice for the screen is HeLa S3 suspension cells. These cells are commonly used for proteome-wide studies of nuclear protein composition and dynamics. To compare cells with the same genetic background in the pilot screen studies, we will treat HeLa cells with shRNAs (Thermo Scientific) to generate a phenotypic spectrum of nuclear variants in HeLa by knockdown of lamin A and emerin. We expect that emerin-knockdown cells will exhibit a less marked difference in deformability than the lamin A-knockdown cells. We will also generate lamin B1 knockdown cells; this nuclear protein has no observable effect on nuclear mechanical properties 19, and thus will serve as a negative control. Together with the mock- and nonmodified control cell lines, these variants should show differential passage times. We will independently confirm the deformability and mechanical properties of these cells and nuclei using microfluidics and micropipette aspiration, and then subject the cells to HR-HTMS. These experiments can establishing HR-HTMS dynamic range and sensitivity for RNAi mechanical screening.

### Example 4

### RNAi Screening

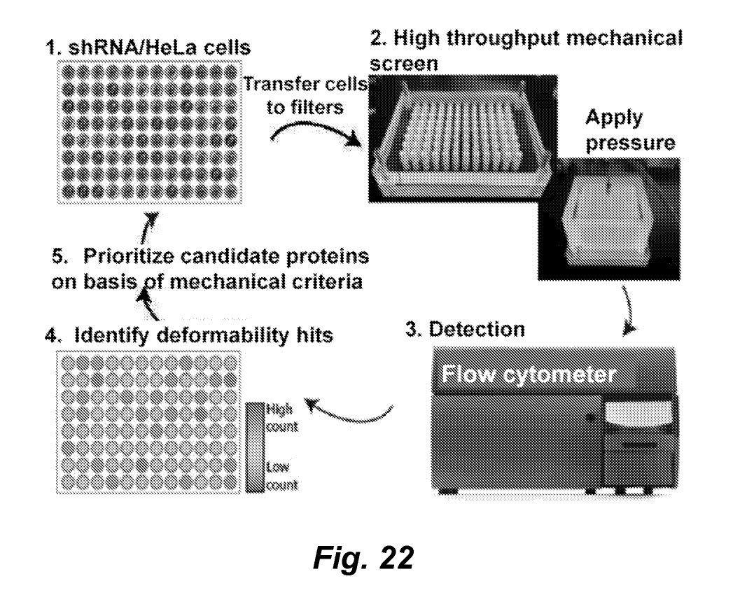

FIG. 22 illustrates the use of the HTMS in an shRNA screen to identify genes that alter mechanical properties of cells. Using liquid-handling robots, the HeLa cells are placed in the wells of a 384-well plate that contains the shRNAs. After incubation and transfer to HR-HTMS, pressure is applied for a defined period of time, and the number of cells that have passaged through the PDMS pores is quantified by flow cytometry. In order to probe an entire shRNA library covering 18,000 genes, process is repeated for about 50 plates. Each plate can contain a row of mock-modified controls, so that the data can be normalized for each plate. The resulting deformability ‘hits’ the variants that show the largest differences in number of passaged cells compared to the negative controls provides a the priority subset of proteins of interest. The results of the screen facilitates identification of major proteins that contribute to the observed nuclear ‘stiffness’ phenotype.

### Example 5

### High Throughput Mechanical Screening (HTMS)

### Basic Characterization using Human Promyelocytic Leukemia (HL60) Cells

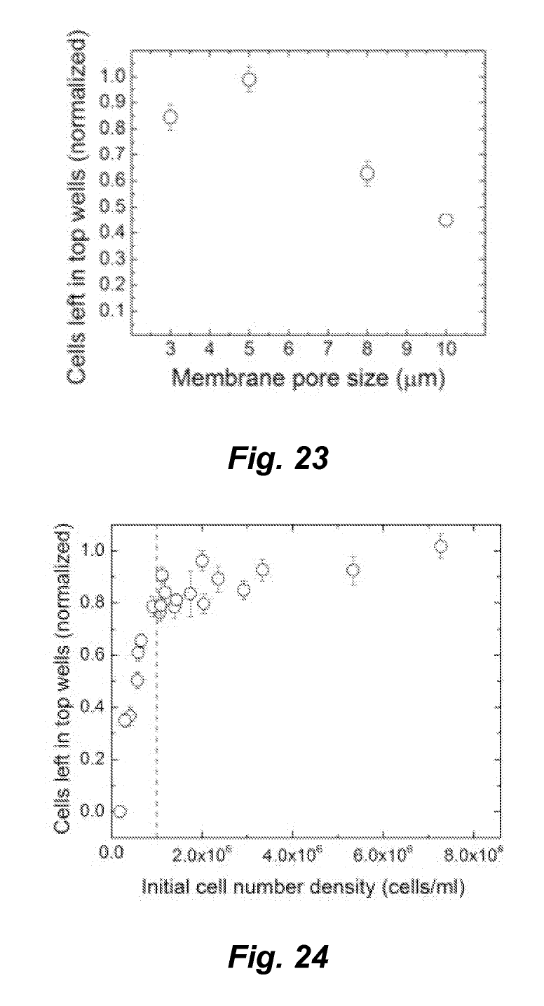

FIG. 23 illustrates the effect of pore size on cell passage in one HTMS device using HL60 cells. In these experiments, it was determined that when rₚₒᵣₑ/r_{cell} was <˜2.5 no apparent filtration was observed indicating that in certain embodiments, it is generally desirable to keep the rₚₒᵣₑ/r_{cell} higher. Thus, in some embodiments, the r_{cell}/rₚₒᵣₑ ratio is greater than about 2.6, or greater than about 2.8, or greater than about 3, or greater than about 3.2, or greater than about 3.4, or greater than about 3.6, or greater than about 3.8, or greater than about 4, or greater than about 4.5, or 5, or 6.

The optimum cell density for use in a particular HTMS device and/or with a particular cell type can readily be determined as illustrated in FIG. 24. It is observed that as cell density increases beyond a particular threshold (see, e.g., dotted line in FIG. 24) cells are retained in the top wells due to pore clogging.

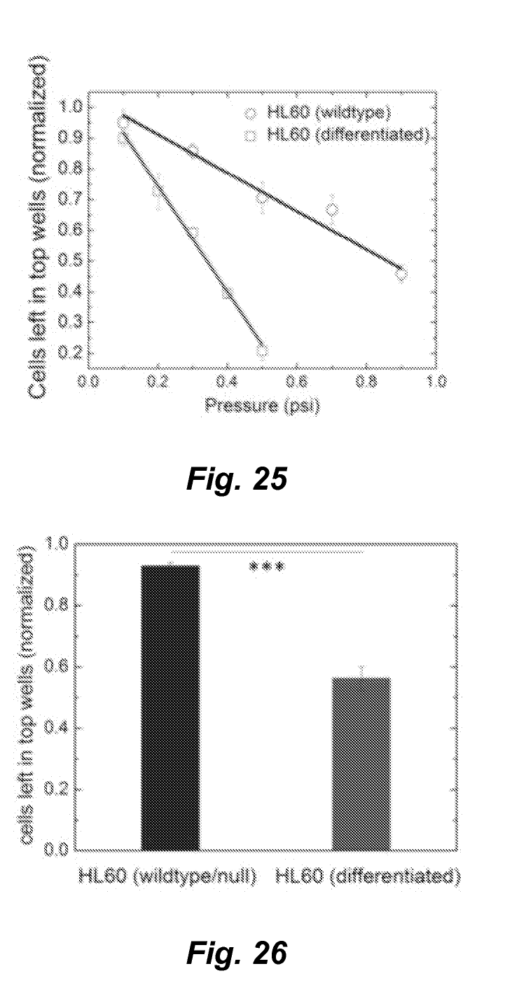

As indicated above, and illustrated in FIG. 25, different cells can have different deformability characteristics. As shown in FIG. 25 HL60 cells and neutrophil-type (ATRA-treated) cells show different pressure dependence in an HTMS system. However, FIG. 26 shows that differences in the mechanical properties of these cells can be detected at a fixed pressure, in this instance, 0.3 psi applied for 20 seconds.

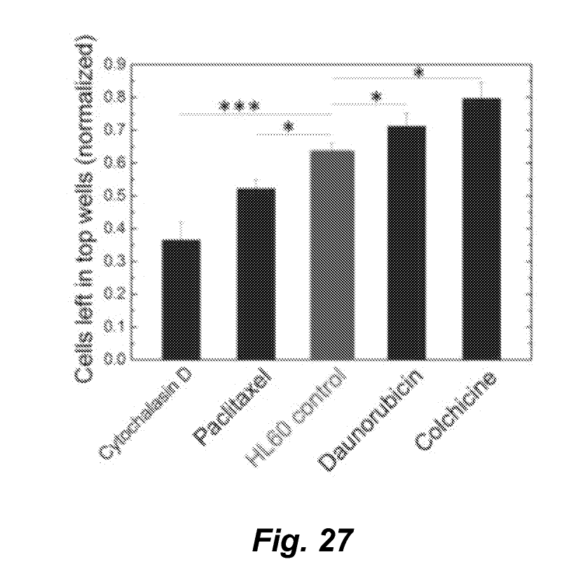

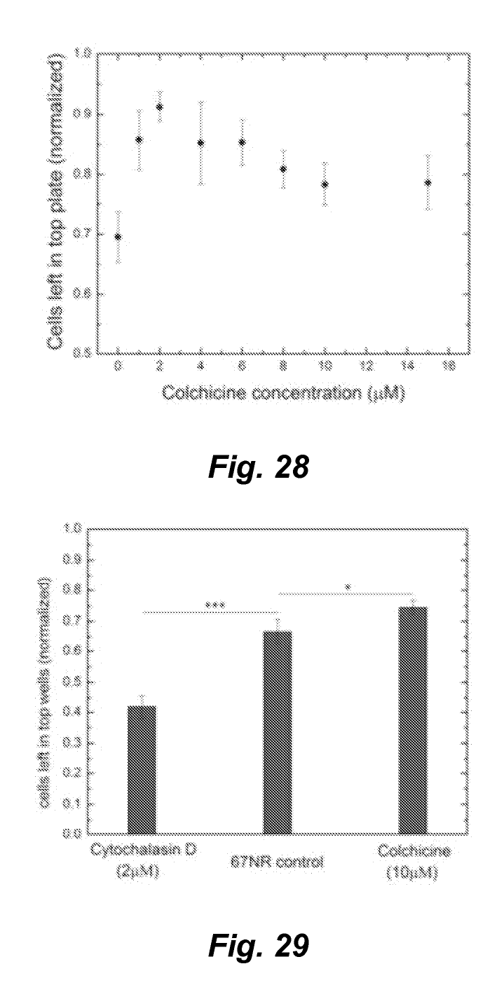

As shown in FIG. 27 the effect of various anti-cancer drugs (e.g., cytochalisin D, paclitaxel, daunorubicin, colchicine) can readily be detected using the HTMS systems described herein. As shown in this Figure, 2.0 μM cytochalasin D and 1.5 μM paclitaxel increase cell deformability, while 1.0 μM dauorubicin and 10.0 μM colchicine decrease the deformability of HL60 cells. Without being bound by a particular theory, it is believed that that the changes in mechanical properties are produced by the action of the drugs on cytoskeletal elements (e.g., actin filaments). Similarly, as shown in FIG. 28 the HTMS measurements made herein can be used to detect dose-response effects of various agents (e.g., anti-cancer drugs) on cells. In this experiment, HL60 cells were treated with 10.0 μM colchicine. Without being bound by a particular theory it is believed that colchicine perturbs microtubules at low concentrations and stabilizes F-actin at high concentrations. The observed increased cell stiffness at low drug concentration is surprising indicating that the system can be used to detect drug effects that may not be predicted or observed using other methods. Dose response effects of colchicine are also observed on adherent murine mammary carcinoma cells (67NR) (see, e.g., FIG. 29).

In some embodiments the HTMS systems described herein can be used to detect the effect of miRNA on cells. In one experiment, miRNA was used to target a gene downstream of p53 and the effect on cell stiffness was determined (see, e.g., FIG. 30).

The miRNA decreases cell proliferation and reverts typical hallmarks of cancer cells. As shown in this figure, treated cells are stiffer compared to cells treated with scrambled control RNA.

In various embodiments the HTMS systems described herein can be used to identify cells that are drug resistant or drug sensitive. As shown in FIG. 31, cisplatin sensitive cells (SKOV3 cells and OVCAR5 cells) are stiffer than cisplatin-resistant cells (SKOV3-CISR cells and OVCAR5-CISR cells). It is believed the HTMS assay for cancer cells offers mechanical characteristics of cells that complements other assays. Moreover, HTMS provides deformability information about a particular sample or cell populations which avoids potential biased sub-population properties of other single-cell methods that assay only a subset of <100 cells.

In addition, the HTMS systems described herein can be used to monitor the progression of drug treatments. Ovarian cancer cells: SKOV3 and OVCAR5 are cisplatin sensitive, while SKOV3-CISR and OVCAR5-CISR are cisplatin resistant. The OVCAR5-CISR cells are OVCAR5 sensitive cells which were treated with cisplatin for >6 months; OVCAR5-CIS+ cells were treated with cisplatin for <1 day and no apparent difference was measured compared with OVCAR5 cells.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A system for the high-throughput screening of mechanical properties of cells and/or particles, said system comprising:
a first structure comprising a plurality of sample receiving wells;
a second structure comprising a plurality of exit ports;
a porous structure disposed between said plurality of sample receiving wells and said plurality of exit ports and in communication with said plurality of sample receiving wells and in communication with said plurality of exit ports, and said first structure, said second structure and said porous structure are sealed or configured such that when sufficient pressure is applied to sample receiving wells in said first structure said cells or particles migrate through the porous structure into said exit ports, and wherein the mean or the median pore size of said porous structure is smaller than the mean or median cross-section of a cell or particle that is to be screened.

2. The system of claim 1, wherein said porous structure is fabricated as a component of said first structure.

3. The system of claim 1, wherein said porous structure is fabricated as a component of said second structure.

4. The system of claim 1, wherein said porous structure is provided as a third structure sandwiched between said first structure said second structure.

5. The system of claim 1, wherein said first structure, said porous structure, and said second structure are fabricated as a single unitary structure.

6. The system according to any one of claims 1-5, wherein said system further comprises a loading plate comprising a plurality of loading wells where said loading plate is disposed on said first structure to align loading wells with said sample receiving wells and permit transfer of one or more samples from the wells comprising said loading plate to wells comprising said plurality of sample receiving wells.

7. The system according to any one of claims 1-6, wherein said system further comprises one or a plurality of micropipette tips where said tips are each inserted into one of the wells comprising said plurality of sample receiving wells.

8. The system according to any one of claims 1-7, wherein there is substantially no gas or fluid leakage between the first structure, the porous structure, and the second structure other than from the receiving wells, through the porous structure, into the exit ports.

9. The system according to any one of claims 1-8, wherein said first structure comprises at least 48 receiving wells.

10. The system according to any one of claims 1-8, wherein said first structure comprises at least 96 receiving wells.

11. The system according to any one of claims 1-8, wherein said first structure comprises at least 384 receiving wells.

12. The system according to any one of claims 1-11, wherein said first structure is fabricated from a material selected from the group consisting of a plastic, a metal or metalloid (such as silicon), synthetic or natural polymers (nitrocellulose, cellulose esters, polyethylene and polypropylene (PE and PP), polytetrafluoroethylene (PTFE)), an elastomeric material, a soft lithography material, a ceramic, and glass.

13. The system according to any one of claims 1-12, wherein said second structure is fabricated from a material selected from the group consisting of a plastic, a metal or metalloid (such as silicon), synthetic or natural polymers (nitrocellulose, cellulose esters, polyethylene and polypropylene (PE and PP), polytetrafluoroethylene (PTFE)), an elastomeric material, a soft lithography material, a ceramic, and glass.

14. The system according to any one of claims 1-13, wherein said porous structure provides an average or median pore size that is substantially uniform with respect to different sample receiving wells.

15. The system according to any one of claims 1-13, wherein said porous structure provides an average or median pore size that is heterogeneous with respect to different sample receiving wells such that different sample receiving wells are in communication with a porous structure of different average or median pore size.

16. The system of claim 15, wherein said porous structure provides a gradient in average or median pore size across the plurality of sample receiving wells.

17. The system according to any one of claims 1-16, wherein said porous structure is fabricated from a material selected from the group consisting of a plastic, a metal or metalloid (such as silicon), synthetic or natural polymers (nitrocellulose, cellulose esters, polyethylene and polypropylene (PE and PP), polytetrafluoroethylene (PTFE)), an elastomeric material, a soft lithography material, a ceramic, and glass.

18. The system according to any one of claims 1-17, wherein said porous structure comprises a porous membrane.

19. The system according to any one of claims 1-17, wherein said porous structure is fabricated from a soft lithography material.

20. The system of claim 19, wherein said soft lithography material comprises a material selected from the group consisting of polydimethylsiloxane (PDMS), polyolefin plastomers (POPs), perfluoropolyethylene (a-PFPE), polyurethane, polyimides, and cross-linked NOVOLAC® (phenol formaldehyde polymer) resin.

21. The system of claim 19, wherein said soft lithography material comprises PDMS.

22. The system according to any one of claims 1-17, and 19-20, wherein said porous structure comprises an array of posts.

23. The system according to any one of claims 1-17, and 19-20, wherein said porous structure comprises a collection of channels.

24. The system according to any one of claims 1-17, and 19-20, wherein said porous structure comprises a bed of particles.

25. The system according to any one of claims 1-24, wherein said first structure is fabricated from a soft lithography material.

26. The system according to any one of claims 1-25, wherein said second structure is fabricated from a soft lithography material.

27. The system according to any one of claims 1-26, wherein said soft lithography material comprises a material selected from the group consisting of polydimethylsiloxane (PDMS), polyolefin plastomers (POPs), perfluoropolyethylene (a-PFPE), polyurethane, polyimides, and cross-linked NOVOLAC® (phenol formaldehyde polymer) resin.

28. The system of claim 27, wherein said soft lithography material comprises PDMS.

29. The system according to any one of claims 1-11, wherein said porous structure comprises a mean pore size of about 1 μm to about 50 μm.

30. The system according to any one of claims 1-11, wherein said porous structure comprises a mean pore size of about 2 μm to about 30 μm.

31. The system according to any one of claims 1-11, wherein said porous structure comprises a mean pore size of about 3 μm to about 10 μm.

32. The system according to any one of claims 1-11, wherein said porous structure comprises a mean pore size of about 5 μm.

33. The system according to any one of claims 1-32, wherein said system further comprises a lid comprising a port to permit entry of a pressurized liquid or gas, wherein said lid is sealed to said first structure to permit pressurization wells comprising said first plurality of wells.

34. The system of claim 33, wherein said wells are pressurized by a gas.

35. The system of claim 34, wherein the gas pressure is regulated by an electro-pneumatic pressure regulator, or some other source of pressure regulation (e.g. manometer)

36. The system of claim 35, wherein said pressure regulator is under computer control.

37. The system according to any one of claims 1-36, wherein sample receiving wells in said first structure contain eukaryotic cells.

38. The system of claim 37, wherein sample receiving wells in said first structure contain mammalian cells.

39. The system of claim 37, wherein sample receiving wells in said first structure contain human cells.

40. The system according to any one of claims 1-36, wherein sample receiving wells in said first structure contain prokaryotic cells.

41. The system according to any one of claims 1-36, wherein sample receiving wells in said first structure contain cells selected from the group consisting of yeast cells, fungal cells, insect cells, bacterial cells, algal cells, plant cells, and mammalian cells.

42. The system according to any one of claims 37-41, wherein sample receiving wells in said first structure are coated with bovine serum albumin (BSA).

43. The system according to any one of claims 1-42, wherein sample receiving wells in said first structure are coated with a surfactant.

44. The system of claim 43, wherein said surfactant is Pluronic F127.

45. The system according to any one of claims 37-42, wherein different sample receiving wells contain different test agents.

46. The system of claim 45, wherein said test agents comprise shRNA, or a small organic molecule.

47. A method of detecting differences in the mechanical properties of cells or particles, said method comprising:
providing a system for the high-throughput screening of mechanical properties of cells and/or particles according to any one of claims 1-44;
placing said cells or particles in wells comprising said plurality of sample receiving wells;
pressurizing the wells containing said cells or particles;
collecting and/or detecting cells or particles that pass through said porous structure into said receiving ports and/or collecting and/or detecting cells or particles that are retained in said first plurality of wells; and
quantifying the number of cells or particles that pass through said porous structure into said exit ports and/or that are retained in said receiving wells where the number of cells or particles retained in the receiving wells and/or that pass through into the exit ports provides a measure of the stiffness of said cells or particles.

48. The method of claim 47, wherein said quantifying comprises quantifying the number of cells or particles that pass through said porous structure into or through said exit ports.

49. The method of claim 47, wherein said quantifying comprises quantifying the number of cells or particles that are retained in said sample receiving wells.

50. The method according to any one of claims 47-49, wherein said pressurizing comprises placing a lid comprising a port to permit entry of a pressurized liquid or gas over wells comprising the sample receiving wells, wherein said lid is sealed to said first structure to permit pressurization of said sample receiving wells.

51. The method of claim 50, wherein said wells are pressurized by a gas.

52. The method of claim 51, wherein the gas pressure is regulated by an electro-pneumatic pressure regulator.

53. The method of claim 52, wherein said pressure regulator is under computer control.

54. The method according to any one of claims 47-53, wherein said cells or particles comprise cells.

55. The method of claim 54, wherein said cells comprise eukaryotic cells.

56. The method of claim 54, wherein said cells or particles comprise mammalian cells.

57. The method of claim 54, wherein said cells comprise human cells.

58. The method of claim 54, wherein said cells comprise cancer cells.

59. The method of claim 54, wherein said cells comprise prokaryotic cells.

60. The method of claim 59, wherein said cells are bacterial cells.

61. The method of claim 59, wherein said cells are selected from the group consisting of yeast cells, fungal cells, insect cells, bacterial cells, algal cells, plant cells, and mammalian cells.

62. The method according to any one of claims 54-61, wherein wells comprising the plurality of sample receiving wells are coated with bovine serum albumin (BSA).

63. The method according to any one of claims 47-62, wherein wells comprising the plurality of sample receiving wells are coated with a surfactant.

64. The method of claim 63, wherein said surfactant is Pluronic F127.

65. The method according to any one of claims 47-64, wherein different sample receiving wells contain different test agents or different combinations of test agents.

66. The method of claim 65, wherein said test agents comprise shRNA, or a small organic molecule.

67. The method of claim 65, wherein said test agents comprise pharmaceuticals.

68. The method of claim 67, wherein said test agents comprise anti-cancer pharmaceuticals or compounds believed to have anti-cancer activity.

69. The method according to any one of claims 47-68, wherein said quantifying comprises utilizing a flow cytometer to quantify the number of cells in in sample receiving wells, and/or that pass through the exit ports or are in wells fed by the exit ports.

70. The method of claim 69, wherein said flow cytometer detects a fluorescent marker in said cells.

71. The method according to any one of claims 47-68, wherein said quantifying comprises utilizing a microplate reader to quantify the number of cells in sample receiving wells, and/or that pass through the exit ports or are in wells fed by the exit ports.

72. The method according to any one of claims 47-68, wherein said quantifying comprises utilizing an image analysis system to quantify the number of cells in wells comprising said plurality of sample receiving wells and/or that pass through the exit ports or are in wells fed by the exit ports.

73. A method determining that cells are responsive or non-responsive to a drug, said method comprising:
providing a system for the high-throughput screening of mechanical properties of cells and/or particles according to any one of claims 1-44;
placing cells to be tested in wells comprising said plurality of sample receiving wells;
contacting said cells with said drug;
pressurizing the sample receiving wells containing said cells;
collecting and/or detecting cells that pass through said porous structure into said exit ports, and/or collecting and/or detecting cells that are retained in said sample receiving wells; and
quantifying the number of cells that pass through said porous structure into said exit ports and/or quantifying the number of cells that are retained in said sample receiving wells where a difference in the number of cells contacted with said drug retained in the sample receiving wells and/or that pass through into the exit ports as compared to the quantity measured for negative control cells indicates that said cells are responsive to said drug, while a substantial lack of said difference is an indicator that said drug does not alter the mechanical properties of said cells.

74. The method of claim 73, wherein said cells comprise eukaryotic cells.

75. The method of claim 74, wherein said cells comprise mammalian cells.

76. The method of claim 74, wherein said cells comprise human cells.

77. The method of claim 74, wherein said cells comprise stem cells.

78. The method of claim 77, wherein said stem cells are selected from the group consisting of adult stem cells, cord blood stem cells, embryonic stem cells, and induced pluripotent cells (IPSCs).

79. The method of claim 74, wherein said cells comprise cancer cells.

80. The method of claim 74, wherein said cells are derived from a tumor in a subject being evaluated for or under treatment for a cancer.

81. The method according to any one of claims 79-80, wherein said cancer comprises a cancer selected from the group consisting of acute lymphoblastic leukemia (ALL), acute myeloid leukemia (AML), Adrenocortical carcinoma, AIDS-related cancers (e.g., kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, brain and spinal cord tumors, brain stem glioma, central nervous system atypical teratoid/rhabdoid tumor, central nervous system embryonal tumors, central nervous system germ cell tumors, craniopharyngioma, ependymoma, breast cancer, bronchial tumors, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm's tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer , pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

82. The method according to any one of claims 74-81, wherein said contacting comprises placing different drugs in different sample receiving wells.

83. The method of claim 82, wherein said drugs comprise anti-cancer pharmaceuticals or compounds believed to have anti-cancer activity.

84. A method of identifying abnormal cells in a subject, said method comprising:
providing a system for the high-throughput screening of mechanical properties of cells and/or particles according to any one of claims 1-37;
placing cells from said subject in wells comprising said plurality of sample receiving wells;
pressurizing the wells containing said cells;
collecting and/or detecting cells that pass through said porous structure into said exit ports, and/or collecting and/or detecting cells that are retained in sample receiving wells; and
quantifying the number of cells that pass through said porous structure into said exit ports and/or quantifying the number of cells that are retained in wells comprising plurality of sample receiving wells where a difference in the number of cells that pass through or that are retained as compared to the same measurement performed on normal healthy cells is an indicator that cells from said subject are abnormal.

85. The method of claim 84, wherein said cells are further screened for one or more cancer markers.

86. The method according to any one of claims 84-85, wherein said cells comprise cells from a non-human mammal.

87. The method according to any one of claims 84-85, wherein said cells comprise cells from a human.

88. The method according to any one of claims 84-87, wherein said cells comprise cells from a tissue biopsy.

89. The method according to any one of claims 84-87, wherein said cells comprise cells from a cell culture.

90. The method according to any one of claims 84-89, wherein said cells are from a mammal diagnosed as having or suspected of having cancer.

91. The method according to any one of claims 84-89, wherein said cells comprise cancer cells.

92. The method according to any one of claims 73-91, wherein said quantifying comprises quantifying the number of cells that pass through said porous structure into said exit ports.

93. The method according to any one of claims 73-91, wherein said quantifying comprises quantifying the number of cells that are retained in wells comprising said plurality of sample receiving wells.

94. A method of enriching a population of cells for cells of particular mechanical properties, said method comprising:
providing a system for the high-throughput screening of mechanical properties of cells and/or particles according to any one of claims 1-37;
placing cells in wells comprising said plurality of sample receiving wells;
pressurizing the wells containing said cells; and
collecting cells that pass through said porous structure into said exit ports, and/or collecting cells that are retained in sample receiving wells;
wherein said collected cells are a population of cells enriched for particular mechanical properties.

95. The method of claim 94, wherein said cells comprise eukaryotic cells.

96. The method of claim 95, wherein cells comprise mammalian cells.

97. The method of claim 96, wherein said cells comprises human cells.

98. The method of claim 94, wherein said cells comprise prokaryotic cells.

99. The method of claim 94, wherein said cells comprise cells selected from the group consisting of yeast cells, fungal cells, insect cells, bacterial cells, algal cells, plant cells, and mammalian cells.

100. The method according to any one of claims 73-99, wherein said pressurizing comprises placing a lid comprising a port to permit entry of a pressurized liquid or gas over cells comprising said plurality sample receiving wells, wherein said lid is sealed to said first layer to permit pressurization wells comprising said plurality of sample receiving wells.

101. The method of claim 100, wherein said wells are pressurized by a gas.

102. The method of claim 101, wherein the gas pressure is regulated by an electro-pneumatic pressure regulator.

103. The method of claim 102, wherein said pressure regulator is under computer control.

104. The method according to any one of claims 73-103, wherein sample receiving wells are coated with bovine serum albumin (BSA).

105. The method according to any one of claims 73-103, wherein sample receiving wells are coated with a surfactant.

106. The method according to any one of claims 73-104, wherein said quantifying comprises utilizing a flow cytometer to quantify the number of cells in wells comprising said plurality of sample receiving wells and/or in that pass out said exit ports.

107. The method of claim 106, wherein said flow cytometer detects a fluorescent marker in said cells.

108. The method according to any one of claims 73-104, wherein said quantifying comprises utilizing a microplate reader to quantify the number of cells in wells comprising said plurality of sample receiving wells and/or that pass through the exit ports.

109. The method according to any one of claims 73-104, wherein said quantifying comprises utilizing an image analysis system to quantify the number of cells in wells comprising said plurality of sample receiving wells and/or that pass into or through the exit ports.
